(19) United States
(12) Patent Application Publication
(10) Pub. No.: US 2011/0059452 A1
(43) Pub. Date: Mar. 10, 2011

(54) METHODS OF SCREENING FOR GASTRIC CANCER

(75) Inventors: James R. Goldenring, Nashville, TN (US); Ki Taek Nam, Franklin, TN (US); Hyuk-Joon Lee, Seoul (KR); Han-Kwang Yang, Seoul (KR); Woo Ho Kim, Seoul (KR)

(73) Assignees: Vanderbilt University; Seoul National University

(21) Appl. No.: 12/858,846

(22) Filed: Aug. 18, 2010

Related U.S. Application Data
(60) Provisional application No. 61/234,869, filed on Aug. 18, 2009.

(51) Int. Cl. C12Q 1/68; G01N 33/574

(52) U.S. Cl. 435/6; 435/7.1

(57) ABSTRACT

Method and systems are provided for diagnosing or monitoring a gastric cancer in a subject. Such methods include providing a biological sample from the subject; determining an amount in the sample of at least one biomarker, selected from the group consisting of: CDH17 and OLFM4; and comparing the amount of the at least one biomarker in the sample, if present, to a control level of the at least one biomarker. Such systems include a probe for selectively binding each of at least one biomarker.

### RELATED APPLICATIONS

This application claims priority from U.S. Provisional Application Ser. No. 61/234,869 filed Aug. 18, 2009, the entire disclosure of which is incorporated herein by this reference.

### GOVERNMENT INTEREST

Subject matter described herein was made with U.S. Government support under Grant Number RO1 DK071590 awarded by the National Institutes of Health (NIH). The government has certain rights in the described subject matter.

### TECHNICAL FIELD

The presently-disclosed subject matter relates to methods for diagnosis and prognosis of gastric cancer in a subject. In particular, the presently-disclosed subject matter relates to diagnostic and prognostic methods based on determining an amount of biomarkers in a biological sample from a subject.

### INTRODUCTION

Although the incidence of gastric cancer has decreased in the western countries, it still ranks as the fourth most common cancer worldwide and the second most common cause of cancer-related death.¹ While considerable improvements have occurred in early detection, surgical technique, and adjuvant chemotherapy,^{2,3} little has been achieved in the development of novel prognostic markers. For prediction of prognosis, only the TNM staging system and surgical curability (R-category) are commonly used in the clinical setting.⁴ Therefore, novel molecular prognostic markers for gastric cancer, especially those with insights within the same TNM stage, are needed not only for the accurate prediction of recurrence, but also for the personalized treatment of each patient. This need is especially apparent in the treatment of early-stage gastric cancer patients, where adjuvant chemotherapy could be applied more selectively if effective prognostic markers were available.

Similar to other malignancies, gene expression profiling using cDNA microarray has been previously performed on tumor samples to identify new diagnostic and prognostic markers for gastric cancer.⁵⁻⁹ Unfortunately, these studies have yielded few useful biomarkers for gastric cancer, likely due to the heterogeneity of the original tumor samples and contamination by the premalignant metaplastic processes in the surrounding mucosa that usually served as the “normal” control. To avoid these problems, the present inventors have focused on gene expression profiling of gastric metaplastic lesions from the gastric cancer patients to identify novel biomarkers affecting the early stage of gastric carcinogenesis.

Intestinal metaplasia (IM) is a well-established precursor in gastric carcinogenesis, especially of intestinal-type tumors.¹⁰ Another metaplastic lesion, designated spasmolytic polypeptide expressing metaplasia (SPEM), shows morphological similarity with deep antral gland cells and expresses trefoil factor 2 (TFF2, spasmolytic polypeptide).^{11,12} In human studies, SPEM was found in 90% of fundic mucosal samples adjacent to gastric cancer.^{13,14} Recent investigations in mice support the development of SPEM from transdifferentiation of normal chief cells into mucous metaplasia following loss of gastric parietal cells.^{15,16} Also, evidence from rodents suggests that IM and dysplasia can develop from SPEM.^{17,18}

A need persists for the development of improved biomarkers and screening methods for gastric cancer.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes method, systems, and kits useful for diagnosing or monitoring a gastric cancer in a subject. Such methods include providing a biological sample from the subject; determining an amount in the sample of at least one biomarker, selected from the group consisting of: CDH17 and OLFM4; and comparing the amount of the at least one biomarker in the sample, if present, to a control level of the at least one biomarker. Such systems include a probe for selectively binding each of at least one biomarker.

In some embodiments, the method includes determining an amount in the sample of a MUC13 biomarker. In some embodiments, the method includes determining an amount in the sample of at least one biomarker, selected from the group consisting of: FABP1, REG4, GDA, DEFA5, ACE2, DMBT1, PCK1, CLCA1, RBP2, KRT20, HSD17B2, MTTP, SLC26A3, SI, ANPEP, LGALS4, SLC5A1, MUC13, SPINK4, APOB, CPS1, GBA3, PRSS7, TFF1, GKN2, TFF2, DPCR1, S100P, FCGBP, LGALS4, CEACAM5, GDA, LYZ, CFTR, MUC5AC, KRT20, ADH1C, AKR1B10, CDCA7, SLC5A1, CYP2C18, ELOVL6, MUC13, SLC6A14, AADAC, HSD17B2, and GCNT3.

In some embodiments, the subject is diagnosed as having the gastric cancer or a risk thereof if there is a measurable difference in the amount of the at least one biomarker in the sample as compared to the control level.

In some embodiments, the method includes selecting a treatment or modifying a treatment for the cancer based on the amount of the at least one biomarker determined.

In some embodiments, the method also includes providing a series of biological samples over a time period from the subject; and determining any measurable change in the amount of the at least one biomarker in each of the biological samples to thereby determine whether to initiate or continue prophylaxis or therapy of the cancer. In some embodiments, the series of biological samples comprises a first biological sample collected prior to initiation of the prophylaxis or treatment for the gastric cancer and a second biological sample collected after initiation of the prophylaxis or treatment.

In some embodiments, the gastric cancer is a precancerous or cancerous pathology selected from the group consisting of intestinal metaplasia (IM), spasmolytic-polypeptide expressing metaplasia (SPEM), a stage I gastric cancer, a stage-II gastric cancer, a stage-III gastric cancer, a stage-IV gastric cancer, a gastric adenocarcinoma, and a node-negative gastric cancer.

In some embodiments, the biological sample includes blood, serum, plasma, gastric secretions, a gastrointestinal biopsy sample, a sample obtained at the time or gastrointestinal resection, microdissected cells from a gastrointestinal biopsy of resection, gastrointestinal cells sloughed into the gastrointestinal lumen, and gastrointestinal cells recovered from stool.

In some embodiments, the amount of the biomarker(s) can be determined by determining an amount of mRNA of the at least one biomarker in the biological sample using an RNA measuring assay; or determining an amount of a polypeptide of the at least one biomarker in the biological sample using a protein measuring assay.

In some embodiments, the RNA measuring assay makes use of an array of RNA hybridization probes or a quantitative polymerase chain reaction assay. In some embodiments, the protein measuring assay makes use of mass spectrometry (MS) analysis, immunoassay analysis, or both. In some embodiments, the immunoassay analysis makes use of one or more antibodies that selectively bind the at least one biomarker.

In some embodiments, determining the amount of the at least one biomarker includes immunohistochemical staining of the at least one biomarker in the biological sample. In some embodiments, the biological sample is selected from a gastrointestinal biopsy sample, a sample obtained at the time of gastrointestinal resection, and microdissected cells from a gastrointestinal biopsy or resection.

In some embodiments, a kit or system is provided for detecting biomarkers of interest, as described herein. The kit can be used for detecting biomarkers with prognostic significance, which can be useful for guiding adjuvant therapy. The kit can be used for diagnosing or monitoring a gastric cancer in a subject. The kit can include a probe for selectively binding each biomarker of interest, as described herein. In some embodiments, the probes are bound to a substrate. In some embodiments, the probes are labeled to allow for detecting the binding of the probes to the at least one biomarker. In some embodiments, the probes are RNA hybridization probes. In some embodiments, the probes are antibodies.

### BRIEF DESCRIPTION OF THE DRAWINGS

FIG. 1. Regions of metaplasia chosen for laser capture microdissection (LCM). Confirmation of the presence of intestinal metaplasia (IM) and spasmolytic polypeptide expressing metaplasia (SPEM) using hematoxylin-eosin staining (A) and double immunohistochemical staining with MUC2 (brown) and TFF2 (red) (B) (original magnification ×40). Arrows indicate IM and arrowheads indicate SPEM in (A).

FIG. 2. Protein expression of the selected genes in metaplastic lineages in the stomach. (A-L) Immunohistochemical staining of the selected genes in normal fundus (left, ×50) and intestinal metaplasia (IM) (right, ×100; insert ×400) (A) ACE2, (B) MUC13, (C) CDH17, (D) OLFM4, (E) MUC5AC, (F) REG4, (G) KRT20, (H) LGALS4, (I) AKR1B10, (J) FABP1, (K) LYZ, (L) DEFA5, (M-O) Immunohistochemical staining of the selected genes in SPEM (M) OLFM4 in SPEM (left, ×50; insert, ×200), (N) LYZ in normal jejunum (left, ×100) and in SPEM (right, ×100; insert, ×400), (O) DPCR1 in normal fundus (left, ×50) and in SPEM (right, ×100; insert ×400).

FIG. 3. Protein expression of the selected genes in gastric adenocarcinoma. (original magnification ×100; all gastric cancer tissues are intestinal-type, except (B) which is diffuse-type). (A) MUC13, membranous pattern, (B) MUC13, cytoplasmic pattern, (C) OLFM4, (D) CDH17, (E) KRT20, (F) MUC5AC, (G) LGALS4, (H) AKR1B10, (I) REG4.

FIG. 4. Disease-specific survival curves of gastric cancer patients according to the expression of CDH17 in a test set and in a validation set. (A) CDH17 in all stages in the test set), (B) CDH17 in all stages in the validation set, (C) CDH17 in curatively resected, stage I cases in the test set, (D) CDH17 in curatively resected, stage I cases in the validation set, (E) CDH17 in curatively resected, node-negative cases in the test set, (F) CDH17 in curatively resected, node-negative cases in the validation set.

FIG. 5. Disease-specific survival curves of gastric cancer patients according to the expression of MUC13 in a test set and in a validation set. (A) membranous pattern of MUC13 in all stages in the test set, (B) membranous pattern of MUC13 in all stages in the validation set), (C) cytoplasmic pattern of MUC13 in all stages in the test set, (D) cytoplasmic pattern of MUC13 in all stages in the validation set).

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The presently-disclosed subject matter provides methods and systems for diagnosis and monitoring of a gastric cancer.

As used herein, “gastric cancer” refers to relevant precancerous or cancerous pathologies. As such, the term is inclusive of premalignant conditions associated with gastric cancer, such as intestinal metaplasia (IM) and spasmolytic-polypeptide expressing metaplasia (SPEM). As will be understood by those skilled in the art, a precancerous pathology can be relevant in screening for a gastric cancer, and/or identifying a increased risk of developing an early or later-stage cancer, and/or making a prognosis, and/or developing a treatment plan. The term “gastric cancer” is further inclusive of early stage gastric cancer, such as a stage I gastric cancer, or a later-stage cancer, such as a stage-II, -III, or -IV gastric cancer. The term “gastric cancer” is further inclusive of a gastric adenocarcinoma, including node-negative gastric adenocarcinoma.

The presently-disclosed subject matter includes methods and systems for diagnosing a gastric cancer in a subject, and for determining whether to initiate or continue prophylaxis or treatment of a gastric cancer in a subject, by identifying at least one biomarker in a biological sample from a subject.

Exemplary biomarkers associated with gastric cancer that can be used in the methods disclosed herein include, but are not limited to, CDH17 and OLFM4, as well as the others set forth in the following tables:

intestinal metaplasia (IM)

| Biomarker | | |
|---|---|---|
| Symbol | Biomarker Name | UniGene ID |
| FABP1 | fatty acid binding protein 1, liver | Hs.380135 |
| REG4 | regenerating islet-derived family, member 4 | Hs.660883 |
| OLFM4 | olfactomedin 4 | Hs.508113 |
| GDA | guanine deaminase | Hs.494163 |
| DEFA5 | defensin, alpha 5, Paneth cell-specific | Hs.655233 |
| ACE2 | angiotensin I converting enzyme (peptidyl-dipeptidase A) 2 | Hs.178098 |
| DMBT1 | deleted in malignant brain tumors 1 | Hs.279611 |
| PCK1 | phosphoenolpyruvate carboxykinase 1 | Hs.1872 |
| CLCA1 | Chloride channel accessory 1 | Hs.194659 |
| RBP2 | retinol binding protein 2, cellular | Hs.655516 |
| KRT20 | keratin 20 | Hs.84905 |
| HSD17B2 | hydroxysteroid (17-β) dehydrogenase 2 | Hs.162795 |
| MTTP | microsomal triglyceride transfer protein | Hs.195799 |
| CDH17 | cadherin 17, LI cadherin (liver-intestine) | Hs.591853 |
| SLC26A3 | solute carrier family 26, member 3 | Hs.1650 |
| SI | sucrase-isomaltase (alpha-glucosidase) | Hs.429596 |
| ANPEP | alanyl (membrane) aminopeptidase | Hs.1239 |
| LGALS4 | lectin, galactoside-binding, soluble, 4 (galectin 4) | Hs.5302 |
| SLC5A1 | solute carrier family 5 (sodium/glucose cotransporter), | Hs.1964 |
| | member 1 | |
| MUC13 | mucin 13, cell surface associated | Hs.5940 |
| SPINK4 | serine peptidase inhibitor, Kazal type 4 | Hs.555934 |
| APOB | apolipoprotein B (including Ag(x) antigen) | Hs.120759 |
| CPS1 | carbamoyl-phosphate synthetase 1, mitochondrial | Hs.149252 |
| GBA3 | glucosidase, beta, acid 3 (cytosolic) | Hs.653107 |
| PRSS7 | protease, serine, 7 (enterokinase) | Hs.149473 |

spasmolytic polypeptide expressing metaplasia (SPEM)

| Biomarker | | |
|---|---|---|
| Symbol | Biomarker Name | UniGene ID |
| OLFM4 | olfactomedin 4 | Hs.508113 |
| TFF1 | trefoil factor 1 | Hs.162807 |
| GKN2 | gastrokine 2 | Hs.16757 |
| TFF2 | trefoil factor 2 (spasmolytic protein 1) | Hs.2979 |
| DPCR1 | diffuse panbronchiolitis critical region 1 | Hs.631993 |
| S100P | S100 calcium binding protein P | Hs.2962 |
| FCGBP | Fc fragment of IgG binding protein | Hs.111732 |
| LGALS4 | lectin, galactoside-binding, soluble, 4 (galectin 4) | Hs.5302 |
| CEACAM5 | carcinoembryonic antigen-related cell adhesion molecule 5 | Hs.709196 |
| GDA | guanine deaminase | Hs.494163 |
| LYZ | lysozyme (renal amyloidosis) | Hs.524579 |
| CFTR | cystic fibrosis transmembrane conductance regulator | Hs.489786 |
| MUC5AC | mucin 5AC, oligomeric mucus/gel-forming | Hs.558950 |
| KRT20 | keratin 20 | Hs.84905 |
| ADH1C | alcohol dehydrogenase 1C (class I), gamma polypeptide | Hs.654537 |
| AKR1B10 | aldo-keto reductase family 1, member B10 (aldose | Hs.116724 |
| | reductase) | |
| CDCA7 | cell division cycle associated 7 | Hs.470654 |
| SLC5A1 | solute carrier family 5 (sodium/glucose cotransporter), | Hs.1964 |
| | member 1 | |
| CYP2C18 | cytochrome P450, family 2, subfamily C, polypeptide 18 | Hs.511872 |
| ELOVL6 | ELOVL family member 6, elongation of long chain fatty | Hs.412939 |
| | acids | |
| MUC13 | mucin 13, cell surface associated | Hs.5940 |
| SLC6A14 | solute carrier family 6 (amino acid transporter), member 14 | Hs.522109 |
| AADAC | arylacetamide deacetylase (esterase) | Hs.506908 |
| HSD17B2 | hydroxysteroid (17-beta) dehydrogenase 2 | Hs.162795 |
| GCNT3 | glucosaminyl (N-acetyl) transferase 3, mucin type | Hs.194710 |

It is noted that the biomarkers disclosed herein are not limited to human biomarkers, or even mRNA biomarkers only. Rather, the present subject matter encompasses biomarkers across animal species that are associated with gastric cancers. In addition, standard gene/protein nomenclature guidelines generally stipulate human gene name abbreviations are capitalized and italicized and protein name abbreviations are capitalized, but not italicized. Further, standard gene/protein nomenclature guidelines generally stipulate mouse, rat, and chicken gene name abbreviations italicized with the first letter only capitalized and protein name abbreviations capitalized, but not italicized. In contrast, the gene/protein nomenclature used herein when referencing specific biomarkers uses all capital letters for the biomarker abbreviation, but is intended to be inclusive of genes (including mRNAs and cDNAs) and proteins across animal species.

A “biomarker” is a molecule useful as an indicator of a biologic state in a subject. With reference to the present subject matter, the biomarkers disclosed herein can be polypeptides that exhibit a change in expression or state, which can be correlated with the risk of developing, the presence of, or the progression of gastric cancers in a subject. In addition, the biomarkers disclosed herein are inclusive of messenger RNAs (mRNAs) encoding the biomarker polypeptides, as measurement of a change in expression of an mRNA can be correlated with changes in expression of the polypeptide encoded by the mRNA. As such, determining an amount of a biomarker in a biological sample is inclusive of determining an amount of a polypeptide biomarker and/or an amount of an mRNA encoding the polypeptide biomarker either by direct or indirect (e.g., by measure of a complementary DNA (cDNA) synthesized from the mRNA) measure of the mRNA.

The terms “polypeptide”, “protein”, and “peptide”, which are used interchangeably herein, refer to a polymer of the 20 protein amino acids, including modified amino acids (e.g., phosphorylated, glycated, etc.), regardless of size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein”, “polypeptide” and “peptide” are used interchangeably herein when referring to a gene product and fragments thereof. Thus, exemplary polypeptides include gene products, naturally occurring proteins, homo logs, orthologs, paralogs, fragments and other equivalents, variants, and fragments of the foregoing.

The terms “polypeptide fragment” or “fragment”, when used in reference to a polypeptide, refers to a polypeptide in which amino acid residues are absent as compared to the full-length polypeptide itself, but where the remaining amino acid sequence is usually identical to the corresponding positions in the reference polypeptide. Such deletions can occur at the amino-terminus or carboxy-terminus of the reference polypeptide, or alternatively both.

A fragment can retain one or more of the biological activities of the reference polypeptide. In some embodiments, a fragment can comprise a domain or feature, and optionally additional amino acids on one or both sides of the domain or feature, which additional amino acids can number from 5, 10, 15, 20, 30, 40, 50, or up to 100 or more residues. Further, fragments can include a sub-fragment of a specific region, which sub-fragment retains a function of the region from which it is derived. When the term “peptide” is used herein, it is intended to include the full-length peptide as well as fragments of the peptide. Thus, an identified fragment of a peptide (e.g., by mass spectrometry or immunoassay) is intended to encompass the fragment as well as the full-length peptide. As such, determining an amount of a biomarker in a sample can include determining an amount of the full-length biomarker polypeptide, modified variants, and/or fragments thereof.

In some embodiments of the presently-disclosed subject matter, a method for diagnosing a gastric cancer in a subject is provided. The terms “diagnosing” and “diagnosis” as used herein refer to methods by which the skilled artisan can estimate and even determine whether or not a subject is suffering from a given disease or condition. The skilled artisan often makes a diagnosis on the basis of one or more diagnostic indicators, such as for example a biomarker, the amount (including presence or absence) of which is indicative of the presence, severity, or absence of the condition.

Along with diagnosis, clinical cancer prognosis is also an area of great concern and interest. It is important to know the aggressiveness of the cancer cells and the likelihood of tumor recurrence in order to plan the most effective therapy. If a more accurate prognosis can be made or even a potential risk for developing the cancer assessed, appropriate therapy, and in some instances less severe therapy for the patient can be chosen. Measurement of cancer biomarkers can be useful in order to separate subjects with good prognosis and/or low risk of developing cancer who will need no therapy or limited therapy from those more likely to develop cancer or suffer a recurrence of cancer who might benefit from more intensive treatments.

As such, “making a diagnosis” or “diagnosing”, as used herein, is further inclusive of determining a risk of developing cancer or determining a prognosis, which can provide for predicting a clinical outcome (with or without medical treatment), selecting an appropriate treatment (or whether treatment would be effective), or monitoring a current treatment and potentially changing the treatment, based on the measure of the diagnostic biomarkers disclosed herein. Further, in some embodiments of the presently disclosed subject matter, multiple determination of the biomarkers over time can be made to facilitate diagnosis and/or prognosis. A temporal change in the biomarker can be used to predict a clinical outcome, monitor the progression of the gastric cancer and/or efficacy of appropriate therapies directed against the cancer. In such an embodiment for example, one might expect to see a decrease in the amount of one or more biomarkers disclosed herein in a biological sample over time during the course of effective therapy.

The presently disclosed subject matter further provides in some embodiments a method for determining whether to initiate or continue prophylaxis or treatment of a cancer in a subject. In some embodiments, the method comprises providing a series of biological samples over a time period from the subject; analyzing the series of biological samples to determine an amount of at least one biomarker disclosed herein in each of the biological samples; and comparing any measurable change in the amounts of one or more of the biomarkers in each of the biological samples. Any changes in the amounts of biomarkers over the time period can be used to predict risk of developing cancer, predict clinical outcome, determine whether to initiate or continue the prophylaxis or therapy of the cancer, and whether a current therapy is effectively treating the cancer. For example, a first time, point can be selected prior to initiation of a treatment and a second time point can be selected at some time after initiation of the treatment. Biomarker levels can be measured in each of the samples taken from different time points and qualitative and/or quantitative differences noted. A change in the amounts of the biomarker levels from the different samples can be correlated with gastric cancer risk, prognosis, determining treatment efficacy, and/or progression of the cancer in the subject.

The terms “correlated” and “correlating,” as used herein in reference to the use of diagnostic and prognostic the biomarkers disclosed herein, refers to comparing the presence or quantity of the biomarker in a subject to its presence or quantity in subjects known to suffer from, or known to be at risk of, a given condition (e.g., a gastric cancer); or in subjects known to be free of a given condition, i.e. “normal subjects” or “control subjects”. For example, a level of one or more biomarkers disclosed herein in a biological sample can be compared to a biomarker levels determined to be associated with a specific type of cancer. The sample's biomarker level is said to have been correlated with a diagnosis; that is, the skilled artisan can use the biomarker level to determine whether the subject suffers from a specific type of cancer, and respond accordingly. Alternatively, the sample's biomarker level can be compared to a control biomarker level known to be associated with a good outcome (e.g., the absence of cancer), such as an average level found in a population of normal subjects.

In certain embodiments, a diagnostic or prognostic biomarker is correlated to a condition or disease by merely its presence or absence. In other embodiments, a threshold level of a diagnostic or prognostic biomarker can be established, and the level of the indicator in a subject sample can simply be compared to the threshold level.

As noted, in some embodiments, multiple determinations of one or more diagnostic or prognostic biomarkers can be made, and a temporal change in the marker can be used to determine a diagnosis or prognosis. For example, a diagnostic marker can be determined at an initial time, and again at a second time. In such embodiments, an increase in the marker from the initial time to the second time can be diagnostic of a particular type or severity of cancer, or a given prognosis. Likewise, a decrease in the marker from the initial time to the second time can be indicative of a particular type or severity of cancer, or a given prognosis. Furthermore, the degree of change of one or more markers can be related to the severity of the cancer and future adverse events.

The skilled artisan will understand that, while in certain embodiments comparative measurements can be made of the same biomarker at multiple time points, one can also measure a given biomarker at one time point, and a second biomarker at a second time point, and a comparison of these markers can provide diagnostic information.

The phrase “determining the prognosis” as used herein refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the presence, absence or levels of a biomarker. Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., not expressing the biomarker or expressing it at a reduced level), the chance of a given outcome (e.g., suffering from a gastric cancer) may be very low (e.g., <1%), or even absent. In contrast, in individuals exhibiting the condition (e.g., expressing the biomarker or expressing it at a level greatly increased over a control level), the chance of a given outcome (e.g., suffering from a gastric cancer) may be high. In certain embodiments, a prognosis is about a 5% chance of a given expected outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance.

The skilled artisan will understand that associating a prognostic indicator with a predisposition to an adverse outcome is a statistical analysis. For example, a biomarker level (e.g., quantity of a biomarker in a sample) of greater than a control level in some embodiments can signal that a subject is more likely to suffer from a cancer than subjects with a level less than or equal to the control level, as determined by a level of statistical significance. Additionally, a change in marker concentration from baseline levels can be reflective of subject prognosis, and the degree of change in marker level can be related to the severity of adverse events. Statistical significance is often determined by comparing two or more populations, and determining a confidence interval and/or a p value. See, e.g., Dowdy and Wearden, Statistics for Research, John Wiley & Sons, New. York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

In other embodiments, a threshold degree of change in the level of a prognostic or diagnostic biomarker disclosed herein can be established, and the degree of change in the level of the indicator in a biological sample can simply be compared to the threshold degree of change in the level. A preferred threshold change in the level for markers of the presently disclosed subject matter is about 5%, about 10%, about 15%, about 20%, about 25%, about 30%, about 50%, about 75%, about 100%, and about 150%. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

The “amount” of a biomarker determined from a sample refers to a qualitative (e.g., present or not in the measured sample), quantitative (e.g., how much is present), or both measurement of the biomarker. The “control level” is an amount (including the qualitative presence or absence) or range of amounts of the biomarker found in a comparable biological sample in subjects free of a gastric cancer, or at least free of the gastric cancer of interest being tested. As one non-limiting example of calculating the control level, the amount of biomarker present in a normal biological sample can be calculated and extrapolated for whole subjects.

An exemplary non-limiting method of the present subject matter for diagnosing a gastric cancer in a subject is now described. The exemplary method includes: providing a biological sample from the subject; determining an amount of at least one biomarker in the biological sample, where the at least one biomarker is selected from FABP1, REG4, OLFM4, GDA, DEFA5, ACE2, DMBT1, PCK1, CLCA1, RBP2, KRT20, HSD17B2, MTTP, CDH17, SLC26A3, SI, ANPEP, LGALS4, SLC5A1, MUC13, SPINK4, APOB, CPS1, GBA3, PRSS7, OLFM4, TFF1, GKN2, TFF2, DPCR1, S100P, FCGBP, LGALS4, CEACAM5, GDA, LYZ, CFTR, MUC5AC, KRT20, ADH1C, AKR1B10, CDCA7, SLC5A1, CYP2C18, ELOVL6, MUC13, SLC6A14, AADAC, HSD17B2, and GCNT3; and comparing the amount in the sample of the at least one biomarker, if present, to a control level of the at least one biomarker. The subject is then diagnosed as having a gastric cancer if there is a measurable difference in the amount of the at least one biomarker in the sample as compared to the control level.

With regard to the step of providing a biological sample from the subject, different types of biological samples can be provided and used in the exemplary method. For example, a serum, plasma, or blood sample can be provided. For another example, gastric secretions can be provided. For still further examples, the following biological samples can be provided: a gastric biopsy sample (e.g., from the stomach); microdissected cells from a gastric biopsy; gastric cells sloughed into the GI lumen; and gastric cells recovered from stool. Methods for obtaining the preceding samples from a subject are generally known in the art.

Turning now to the step of determining an amount of at least one biomarker in the biological sample, various methods known to those skilled in the art can be used to identify the one or more biomarkers in the provided biological sample. In some embodiments, determining the amount of the at least one biomarker comprises using an RNA measuring assay to measure mRNA encoding biomarker polypeptides in the sample and/or using a protein measuring assay to measure amounts of biomarker polypeptides in the sample.

In certain embodiments of the method, the amounts of biomarkers can be determined by probing for mRNA of the biomarker in the sample using any RNA identification assay known to those skilled in the art. Briefly, RNA can be extracted from the sample, amplified, converted to cDNA, labeled, and allowed to hybridize with probes of a known sequence, such as known RNA hybridization probes (selective for mRNAs encoding biomarker polypeptides) immobilized on a substrate (e.g., an array or microarray) or quantitated by real time PCR (e.g., quantitative real-time PCR, such as available from Bio-Rad Laboratories, Hercules, Calif., U.S.A.). Because the probes to which the nucleic acid molecules of the sample are bound are known, the molecules in the sample can be identified. In this regard, DNA probes for one or more of FABP1, REG4, OLFM4, GDA, DEFA5, ACE2, DMBT1, PCK1, CLCA1, RBP2, KRT20, HSD17B2, MTTP, CDH17, SLC26A3, SI, ANPEP, LGALS4, SLC5A1, MUC13, SPINK4, APOB, CPS1, GBA3, PRSS7, OLFM4, TFF1, GKN2, TFF2, DPCR1, S100P, FCGBP, LGALS4, CEACAM5, GDA, LYZ, CFTR, MUC5AC, KRT20, ADH1C, AKR1B10, CDCA7, SLC5A1, CYP2C18, ELOVL6, MUC13, SLC6A14, AADAC, HSD17B2, and GCNT3 can be immobilized on a substrate and provided for use in practicing a method in accordance with the present subject matter.

With regard to determining amounts of biomarker polypeptides in samples, mass spectrometry and/or immunoassay devices and methods can be used, although other methods are well known to those skilled in the art as well. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference. Immunoassay devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377, each of which is hereby incorporated by reference in its entirety.

Thus, in certain embodiments of the presently-disclosed subject matter, biomarker peptides are analyzed using an immunoassay. The presence or amount of a biomarker peptide disclosed herein can be determined using antibodies or fragments thereof specific for each biomarker polypeptide, or fragment thereof, and detecting specific binding. For example, in some embodiments, the antibody specifically binds FABP1, REG4, OLFM4, GDA, DEFA5, ACE2, DMBT1, PCK1, CLCA1, RBP2, KRT20, HSD17B2, MTTP, CDH17, SLC26A3, SI, ANPEP, LGALS4, SLC5A1, MUC13, SPINK4, APOB, CPS1, GBA3, PRSS7, OLFM4, TFF1, GKN2, TFF2, DPCR1, S100P, FCGBP, LGALS4, CEACAM5, GDA, LYZ, CFTR, MUC5AC, KRT20, ADH1C, AKR1B10, CDCA7, SLC5A1, CYP2C18, ELOVL6, MUC13, SLC6A14, AADAC, HSD17B2, or GCNT3, which is inclusive of antibodies that bind the full-length peptides or a fragment thereof. In some embodiments, the antibody is a monoclonal antibody.

Any suitable immunoassay can be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies or fragments thereof specific for the markers is also contemplated by the presently-disclosed subject matter. The antibodies can be immobilized onto a variety of solid supports, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on a solid support. This strip can then be dipped into the test biological sample and then processed quickly through washes and detection steps to generate a measurable signal, such as for example a colored spot.

In some embodiments, mass spectrometry (MS) analysis can be used alone or in combination with other methods (e.g., immunoassays or RNA measuring assays) to determine the presence and/or quantity of the one or more biomarkers disclosed herein in a biological sample. In some embodiments, the MS analysis comprises matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers. Methods for utilizing MS analysis, including MALDI-TOF MS and ESI-MS, to detect the presence and quantity of biomarker peptides in biological samples are known in the art. See, e.g., U.S. Pat. Nos. 6,925,389; 6,989,100; and 6,890,763 for further guidance, each of which is incorporated herein by this reference.

In some embodiments, the at least one biomarker is assessed using immunohistochemical staining of the provided biological sample or series of samples. In some embodiments, the stained samples are selected from a biopsy sample and a resection sample.

Although certain embodiments of the method only call for a qualitative assessment of the presence or absence of the one or more biomarkers in the biological sample, other embodiments of the method call for a quantitative assessment of the amount of each of the one or more markers in the biological sample. Such quantitative assessments can be made, for example, using one of the above mentioned methods, as will be understood by those skilled in the art.

In certain embodiments of the method, it may be desirable to include a control sample that is analyzed concurrently with the biological sample, such that the results obtained from the biological sample can be compared to the results obtained from the control sample. Additionally, it is contemplated that standard curves can be provided, with which assay results for the biological sample may be compared. Such standard curves present levels of biomarker as a function of assay units, i.e., fluorescent signal intensity, if a fluorescent label is used. Using samples taken from multiple donors, standard curves can be provided for control levels of the one or more biomarkers in normal tissue, as well as for “at-risk” levels of the one or more biomarkers in tissue taken from donors with metaplasia or from donors with gastric cancer.

In certain embodiments of the method, a subject is identified as having metaplasia upon identifying in a biological sample obtained from the subject one or more biomarkers selected from: FABP1, REG4, OLFM4, GDA, DEFA5, ACE2, DMBT1, PCK1, CLCA1, RBP2, KRT20, HSD17B2, MTTP, CDH17, SLC26A3, SI, ANPEP, LGALS4, SLC5A1, MUC13, SPINK4, APOB, CPS1, GBA3, PRSS7, OLFM4, TFF1, GKN2, TFF2, DPCR1, S100P, FCGBP, LGALS4, CEACAM5, GDA, LYZ, CFTR, MUC5AC, KRT20, ADH1C, AKR1B10, CDCA7, SLC5A1, CYP2C18, ELOVL6, MUC13, SLC6A14, AADAC, HSD17B2, and GCNT3. In other embodiments of the method, the identification of one or more of such biomarkers in a biological sample obtained from the subject results in the subject being identified as having cancer.

Regardless of whether the one or more biomarkers are being identified in the biological samples by measuring biomarker gene-expression, e.g., mRNA, or by directly measuring the protein biomarkers, it is contemplated that the efficacy, accuracy, sensitivity, and specificity of the diagnostic method can be enhanced by probing for multiple biomarkers in the biological sample. For example, in certain embodiments of the method, the biological sample can be probed for two or more biomarker selected from: FABP1, REG4, OLFM4, GDA, DEFA5, ACE2, DMBT1, PCK1, CLCA1, RBP2, KRT20, HSD17B2, MTTP, CDH17, SLC26A3, SI, ANPEP, LGALS4, SLC5A1, MUC13, SPINK4, APOB, CPS1, GBA3, PRSS7, OLFM4, TFF1, GKN2, TFF2, DPCR1, SLOOP, FCGBP, LGALS4, CEACAM5, GDA, LYZ, CFTR, MUC5AC, KRT20, ADH1C, AKR1B10, CDCA7, SLC5A1, CYP2C18, ELOVL6, MUC13, SLC6A14, AADAC, HSD17B2, and GCNT3. For another example, the biological sample can be probed for 2-5 biomarkers selected from: FABP1, REG4, OLFM4, GDA, DEFA5, ACE2, DMBT1, PCK1, CLCA1, RBP2, KRT20, HSD17B2, MTTP, CDH17, SLC26A3, SI, ANPEP, LGALS4, SLC5A1, MUC13, SPINK4, APOB, CPS1, GBA3, PRSS7, OLFM4, TFF1, GKN2, TFF2, DPCR1, S100P, FCGBP, LGALS4, CEACAM5, GDA, LYZ, CFTR, MUC5AC, KRT20, ADH1C, AKR1B10, CDCA7, SLC5A1, CYP2C18, ELOVL6, MUC13, SLC6A14, AADAC, HSD17B2, and GCNT3. For another example, the biologic sample can be probed for 6-10 biomarkers selected from: FABP1, REG4, OLFM4, GDA, DEFA5, ACE2, DMBT1, PCK1, CLCA1, RBP2, KRT20, HSD17B2, MTTP, CDH17, SLC26A3, SI, ANPEP, LGALS4, SLC5A1, MUC13, SPINK4, APOB, CPS1, GBA3, PRSS7, OLFM4, TFF1, GKN2, TFF2, DPCR1, S100P, FCGBP, LGALS4, CEACAM5, GDA, LYZ, CFTR, MUC5AC, KRT20, ADH1C, AKR1B10, CDCA7, SLC5A1, CYP2C18, ELOVL6, MUC13, SLC6A14, AADAC, HSD17B2, and GCNT3.

The analysis of markers can be carried out separately or simultaneously with additional markers within one test sample. For example, several markers can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker levels over time. Increases or decreases in marker levels, as well as the absence of change in marker levels, can provide useful information about the disease status that includes, but is not limited to identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

The subject is diagnosed as having a gastric cancer if, when compared to a control level, there is a measurable difference in the amount of the at least one biomarker in the sample. Conversely, when no probed biomarker is identified in the biological sample, the subject can be identified as not having gastric cancer, not being at risk for the cancer, or as having a low risk of the cancer. In this regard, subjects Having the cancer or risk thereof can be differentiated from subjects having low to substantially no cancer or risk thereof. Those subjects having a risk of developing a gastric cancer can be placed on a more intensive and/or regular screening schedule, including upper endoscopic surveillance. On the other hand, those subjects having low to substantially no risk may avoid being subjected to an endoscopy, until such time as a future screening, for example, a screening conducted in accordance with the present subject matter, indicates that a risk of gastric cancer has appeared in those subjects.

As mentioned above, depending on the embodiment of the method of the present subject matter, identification of the one or more biomarkers can be a qualitative determination of the presence or absence of the biomarkers, or it can be a quantitative determination of the concentration of the biomarkers. In this regard, in the exemplary method, the step of diagnosing the subject as having, or at risk of developing, gastric cancer indicates that certain threshold measurements are made, i.e., the levels of the one or more biomarkers in the biological sample exceed predetermined control levels. In certain embodiments of the method, the control level is any detectable level of the biomarker. In other embodiments of the method where a control sample is tested concurrently with the biological sample, the predetermined level is the level of detection in the control sample. In other embodiments of the method, the predetermined level is based upon and/or identified by a standard curve. In other embodiments of the method, the predetermined level is a specifically identified concentration, or concentration range. As such, the predetermined level can be chosen, within acceptable limits that will be apparent to those skilled in the art, based in part on the embodiment of the method being practiced and the desired specificity, etc.

Further with respect to the diagnostic methods of the presently disclosed subject matter, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided in accordance with the presently disclosed subject matter.

As such, the presently disclosed subject matter provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Thus, also provided is the diagnosis and treatment of livestock, including, but not limited to, domesticated swine, ruminants, ungulates, horses (including race horses), and the like.

The presently-disclosed subject matter further includes a system for diagnosing a gastric cancer in a subject. The system can be provided, for example, as a commercial kit that can be used to screen for a risk of gastric cancer or diagnose a gastric cancer in a subject from whom a biological sample has been collected. An exemplary system provided in accordance with the present subject matter includes probes for selectively binding each of one or more biomarkers selected from: FABP1, REG4, OLFM4, GDA, DEFA5, ACE2, DMBT1, PCK1, CLCA1, RBP2, KRT20, HSD17B2, MTTP, CDH17, SLC26A3, SI, ANPEP, LGALS4, SLC5A1, MUC13, SPINK4, APOB, CPS1, GBA3, PRSS7, OLFM4, TFF1, GKN2, TFF2, DPCR1, S100P, FCGBP, LGALS4, CEACAM5, GDA, LYZ, CFTR, MUC5AC, KRT20, ADH1C, AKR1B10, CDCA7, SLC5A1, CYP2C18, ELOVL6, MUC13, SLC6A14, AADAC, HSD17B2, and GCNT3; and components for detecting the binding of the probes to the one or more biomarkers.

In certain embodiments of the system, the probes can be RNA hybridization probes, in which case the RNA of the biological sample would be isolated, amplified, converted to cDNA, labeled, and incubated with the probes to allow for hybridization. The binding of the probes to the cDNA of the biomarkers can be detected using the label of the probe, which can be, for example, a fluorescent label.

In other embodiments of the system, the probes can be antibodies that selectively bind the protein biomarkers. The binding of the antibodies to the biomarkers can be detected, for example, using an enzyme-linked antibody.

The system can also include certain samples for use as controls. The system can further include one or more standard curves providing levels of biomarker mRNA, or levels of biomarker protein as a function of assay units.

Thus, in some embodiments of the presently-disclosed subject matter, a kit for the analysis of biomarkers is provided that comprises probes, including for example antibodies selective for biomarker polypeptides or RNA hybridization probes that can selectively bind mRNA biomarkers (or cDNA amplified therefrom), having specificity for one or more biomarkers disclosed herein. The probes can in some embodiments be bound to a substrate. Such a kit can comprise devices and reagents for the analysis of at least one test sample. The kit can further comprise instructions for using the kit and conducting the analysis. Optionally the kits can contain one or more reagents or devices for converting a marker level to a diagnosis or prognosis of the subject.

The practice of the presently disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples. The following examples may include compilations of data that are representative of data gathered at various times during the course of development and experimentation related to the presently disclosed subject matter.

### EXAMPLES

### Oligonucleotide Microarray from Microdissected RNA

Total RNAs from both IM and SPEM lineages adjacent to intestinal-type gastric cancer in fundus were collected from 6 patients who underwent gastrectomy. In addition, since transdifferentiation of chief cells into SPEM appears to be the first step in metaplastic response to oxyntic atrophy, RNAs from normal chief cells were collected from 6 patients who underwent gastrectomy with no evidence of atrophic gastritis, IM, SPEM or gastric cancer in the fundic mucosa. All samples were obtained from Department of Surgery at Seoul National University Hospital (SNUH) from July 2007 to July 2008. This work was approved by the institutional review board (IRB) at SNUH and written consents were obtained from each patient. Detailed information on each patient is shown in Table A.

TABLE A
Patients' Characteristics for Complementary DNA microarray

| No. | Sex | Age | Diagnosis | WHO | Size (cm) | T | N | M | TNM |
|---|---|---|---|---|---|---|---|---|---|
| C1 | M | 67 | gastric cancer | MD | 1.6 | T2a | N0 | M0 | Ib |
| C2 | F | 59 | gastric cancer | MD | 2.4 | T1 | N0 | M0 | Ia |
| C3 | M | 78 | gastric cancer | PD | 4.2 | T3 | N3 | M0 | IV |
| C4 | F | 75 | gastric cancer | Pap. | 3.2 | T1 | N0 | M0 | Ia |
| C5 | M | 60 | gastric cancer | MD | 3.2 | T2b | N0 | M0 | Ib |
| C6 | M | 67 | gastric cancer | PD | 8.5 | T3 | N1 | M0 | IIIa |
| N1 | M | 63 | GIST | | | | | | |
| N2 | M | 60 | duodenal ulcer | | | | | | |
| N3 | M | 57 | Schwannoma | | | | | | |
| N4 | M | 62 | GIST | | | | | | |
| N5 | M | 50 | GIST | | | | | | |
| N6 | M | 15 | duodenal ulcer | | | | | | |

Abbreviations:
M, male;
F, female;
GIST, gastrointestinal stromal tumor;
WHO, pathologic classification according to World Health Organization;
MD, moderately differentiated;
PD, poorly differentiated;
Pap., papillary adenocarcinoma.

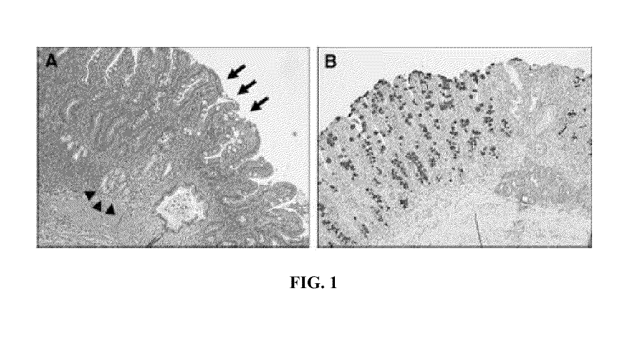

Before performing the laser capture microdissection (LCM), double immunohistochemical staining with anti-human MUC2 (1:200, sc-15334, Santa Cruz, Calif.) and anti-human TFF2/SP (1:100, a gift from Dr. Nicholas Wright, Cancer UK, London, UK) as well as hematoxylin-eosin staining were performed for every tissue sample to confirm the presence and location of IM and SPEM (FIG. 1). LCM procedures were performed using a Veritas Microdissection System (Molecular Devices, CA). Total RNA was extracted and isolated using a Picopure RNA Isolation Kit (Molecular Devices).

Isolated RNAs were amplified using a NuGEN FFPE amplification kit and labeled using a NuGEN Ovation™ cDNA Biotin Module V2 kit (San Carlos, Calif.). RNA quality was determined using the Agilent 2100 bioanalyzer. Five μg of each sample was hybridized to Affymetrix U133 Plus 2.0 GeneChip® Expression arrays (˜55,000 probes) according to manufacturer's instructions. The raw expression data were converted to expression values using the Affy function in R (http://www.bioconductor.org).

Gene Selection

Once expression values were obtained, those probes/features that had at least 25% samples with intensities above 100 fluorescent units and inter-quartile range of at least 0.5 were filtered. The log2-based expression levels were examined using analysis of variance (ANOVA) and ebayes-moderated t-tests implemented in the limma package; the pair-wise contrasts tested chief cell versus IM or SPEM. After type I error was mitigated by first testing for the overall p-value of any difference in means, only those that were found significant in the overall test underwent pair-wise tests. The significant p-values from the two pair-wise tests (chief cell versus IM and chief cell versus SPEM) were ranked and a candidate probe list was compiled, using False Discovery Rate adjusted p-value cut-offs obtained by the linear step-up method described by Benjamini and Hochberg.¹⁹ The Annotate package was used to convert the probe set definitions to searchable forms that were linked to web-based databases. Pathways associated with these candidate probes were examined using the SPIA package. The candidate probes associated with known genes were filtered manually for further analyses of their protein expression. Gene products were prioritized for further analysis based on their classification as (1) secretory or extracellular protein, (2) protein with limited expression in stomach and other tissues, or (3) a novel marker in the oncologic field. Final selection was based on the availability of antibodies for immunohistochemical staining in paraffin-embedded tissues.

Tissue Microarray (TMA) Analysis

To evaluate the protein expression in the normal fundus and metaplastic and cancerous lesions, two small-scale gastric cancer TMAs were used: (1) a collection of 42 gastric adenocarcinomas resected at Vanderbilt University Hospital (Vanderbilt-GC; median age: 67 yrs, M:F=24:18,),²⁰ and (2) a collection of 36 gastric adenocarcinomas resected at SNUH (SNUH-TA78, SuperBioChips, Seoul, Korea; median age: 58 yrs, M:F=27:9,). Another two large-scale sets of tissue microarrays: (1) a collection of 450 gastric adenocarcinomas resected at SNUH in 2004 (SNUH-2004-GC, SuperBioChips) as a test set and (2) a collection of 502 gastric adenocarcinomas resected at SNUH in 1996 (SNUH-1996-GC, SuperBioChips) as a validation set, were used to evaluate the expression profiles of proteins, which were expressed in more than 40% of gastric cancers in initial tissue arrays. In both sets, annotated data for each case were available for age, sex, tumor size and location, Lauren classification, TNM stage (according to 6^{th} UICC/AJCC TNM classification), lymphatic invasion, venous invasion, surgical curability, and disease-specific survival period (Table B). The median follow-up periods were 49.1 months (range: 0.4˜64.4 mo) in SNUH-2004-GC and 76.0 months (range: 2.0˜96.0 mo) in SNUH-1996-GC, respectively.

TABLE B
Information of the 13 selected primary antibodies used in immunohistochemistry

| Antigen* | Antibody (clone) | dilution | source |
|---|---|---|---|
| ACE2 | Rabbit polyclonal (HPA000288) | 1/250 | Sigma-Aldrich, St. Louis, MO |
| AKR1B10 | mouse IgG2a (H4025) | 1/100 | Dr. Hiroyuki Aburatani, University |
| | | | of Tokyo, Japan⁴⁵ |
| CDH17 | mouse IgG1 (ab54511) | 1/250 | Abcam, Cambridge, MA |
| DEFA5 | Rabbit polyclonal (HPA015775) | 1/225 | Sigma-Aldrich, St. Louis, MO |
| DPCR1 | Rabbit polyclonal (HPA014036) | 1/25 | Sigma-Aldrich, St. Louis, MO |
| FABP | Rabbit polyclonal (ab7807) | 1/50 | Abcam, Cambridge, MA |
| KRT20 | mouse IgG2a (N1627) | prediluted | Dako, Glostrup, Denmark |
| LGALS4 | mouse IgG1 (NCL-L-GAL4) | 1/50 | Novocastra, Newcastle, UK |
| LYZ | Rabbit polyclonal (EC 3.2.1.17) | 1/400 | Dako, Glostrup, Denmark |
| MUC5AC | mouse IgG1 (45M1) | 1/100 | Lab Vision, Fremont, CA |
| MUC13 | mouse IgG1 (ppz0020) | 1/500 | Dr. Hiroyuki Aburatani, University |
| | | | of Tokyo, Japan²¹ |
| OLFM4 | Rabbit polyclonal | 1/200 | Dr. Griffin P. Rodgers, NCI, |
| | | | Bethesda, MD²³ |
| REG4 | Goat polyclonal (AF1379) | 1/100 | R&D System, Minneapolis, MN |

*Full titles of abbreviated antigen names are shown in Table E; sorted in alphabetic order.

None of the patients received preoperative chemotherapy or radiotherapy. Extended lymph node dissection was uniformly applied for the curatively resected cases, with mean number of retrieved lymph nodes of 31.5 (in the test set) and 32.0 (in the validation set), respectively. Adjuvant chemotherapy was not indicated in patients with stage Ia, but was usually administered in patients with stage II or higher disease. In patients with stage Ib, adjuvant chemotherapy was selectively indicated considering patient's physical activity and the presence of co-morbidity. A 5-fluorouracil (5-FU) based combination (5-FU plus cisplatin or 5-FU plus mitomycin) was the most commonly used chemotherapeutic regimen. The analysis of survival data of the patient was approved by the IRB at SNUH.

Immunohistochemical Staining

For the immunohistochemistry in human tissues, except SNU-2004-GC and SNU-1996-GC, sections were blocked using normal serum provided in the Vectastain kit (Vector Laboratories, Burlingame, Calif.) and then incubated with the primary antibody overnight at 4° C. After incubation with biotinylated secondary antibody for an hour at room temperature, each slide was incubated either with horseradish-peroxidase-conjugated streptavidin followed by development with diaminobenzidine (Biogenex, San Ramon, Calif.) or with alkaline phosphatase-conjugated streptavidin followed by development with Vector Red (Vector Laboratories). The sections were counterstained with Mayer's hematoxylin. Detailed information on the selected primary antibodies is shown in Table C.

TABLE C
Patients' Demographics of the Test set SNUH-2004-GC and the
Validation Set SNUH-1996-GC Used in This Study

| | SNUH-2004- | | SNUH-1996- | |
|---|---|---|---|---|
| | GC (n = 450) | % | GC (n = 502) | % |
| Sex | | | | |
| Male | 327 | 72.7 | 336 | 66.8 |
| Age, y | | | | |
| Mean ± SD | 57.5 | ±12.6 | 56.8 | ±10.9 |
| Size (cm) | | | | |
| Mean ± SD | 5.5 | ±3.1 | 5.2 | ±2.6 |
| Location | | | | |
| Antral | 220 | 49.0 | 301 | 60.1 |
| Non-antral | 209 | 46.5 | 155 | 30.9 |
| Whole | 20 | 4.5 | 45 | 9.0 |
| Lauren | | | | |
| Intestinal | 185 | 41.1 | 215 | 42.7 |
| Diffuse | 185 | 41.1 | 270 | 53.7 |
| Mixed | 77 | 17.1 | 17 | 3.4 |
| TNM | | | | |
| I | 199 | 44.2 | 184 | 36.6 |
| II | 87 | 19.3 | 117 | 23.3 |
| III | 82 | 18.2 | 123 | 24.5 |
| IV | 82 | 18.2 | 79 | 15.7 |
| R-category | | | | |
| R0 | 403 | 89.6 | 462 | 91.8 |
| R1/2 | 47 | 10.4 | 40 | 8.2 |
| Lymphatic invasion | | | | |
| No | 188 | 41.8 | 332 | 66.0 |
| Yes | 262 | 58.2 | 171 | 34.0 |
| Venous invasion | | | | |
| No | 373 | 82.9 | 475 | 94.4 |
| Yes | 77 | 17.1 | 28 | 5.6 |

SD, standard deviation.

For the immunohistochemical staining of SNU-2004-GC and SNU-1996-GC, an automated procedure was applied with a Bond-Max Immunostainer and a Bond polymer Refine Detection Kit (Leica Microsystems, Germany) according to the manufacturer's recommendations.

After selecting only cancer tissues in each core, pre-defined staining patterns (membranous or cytoplasmic) of each protein were considered positive. A staining intensity was scored as 0 (negative), 1 (positive), and 2 (strong positive), and dichotomized into negative (0) and positive (1-2) for further analysis. If the staining was observed in less than 10% of total cancer cells within a core, it was considered as negative. Each TMA was scored independently by different pathologic specialists without any clinical information (Vanderbilt-GC and SNUH-TA78 by N.K.T., SNUH-2004-GC by P.H.S., SNU-1996-GC by K.M.A. and K.W.H.)

Statistical Analysis of Tissue Array Staining

The association between protein expression and clinicopathologic variables was evaluated using the χ² test. Disease-specific survival curves were calculated by the Kaplan-Meier method, and the log-rank test was used to evaluate the statistical difference. Any clinicopathologic variables as well as the expression of certain proteins with a log-rank p-value less than 0.1 were entered into the multivariate analysis. The Cox proportional hazards model was used for the multivariate analysis to identify independent prognostic factors for survival in a combined cohort of a test and a validation set. In addition, prognostic implications of each protein were evaluated in the subgroup stratified according to tumor location or Lauren classification in a combined cohort. All statistical analyses were conducted using the SPSS version 13.0 (Chicago, Ill., USA).

Gene Expression Profile of SPEM and IM Compared to Normal Chief Cells

Based on the present inventors' recent studies indicating that SPEM is derived from chief cells in mice,^{15,16} the expression profiles for microdissected IM and SPEM were sought to be compared with normal chief cells. 858 probes were identified, which were differentially expressed between chief cells versus IM or SPEM. Among them, 45 probes were significantly up-regulated in both SPEM and IM, 523 were significantly up-regulated in IM alone, 287 were significantly down-regulated in IM alone, and 3 were significantly up-regulated in SPEM alone. No probe was significantly up-regulated in IM and simultaneously significantly down-regulated in SPEM, and vice versa (Table D).

TABLE D
Cox multivariate analysis for disease-specific survival in
subgroups of gastric cancer patients

| | | | | |
|---|---|---|---|---|
| | | No. of | | |
| Factor | Variable | patients | 95% CI | P value |

Curatively resected, stage I gastric cancer (n = 383)

| | | | | |
|---|---|---|---|---|
| TNM stage | la/lb | 208/175 | 2.348-135.597 | .005^{a} |
| Venous Invasion | No/yes | 371/12 | 2.097-69.908 | .005^{a} |
| CDH17^{b} | No/yes | 122/237 | 2.858-38.970 | <.001^{a} |

Curatively resected, node-negative gastric cancer (n = 378)

| | | | | |
|---|---|---|---|---|
| T-stage | T1 | 208 | — | .002^{a} |
| | T2 | 150 | 2.178-133.110 | .007 |
| | T3 | 18 | 0.562-164.628 | .118 |
| | T4 | 2 | 18.452-9976.457 | <.001 |
| CDH17^{b} | No/yes | 123/228 | 1.521-16.108 | .008^{a} |
| MUC13 (memb)^{b} | No/yes | 180/173 | 0.545-5.932 | .335 |
| Size (cm)^{c} | <5/≧5 | 269/103 | 0.282-2.643 | .797 |

^{a}Statistically significant P values (P < .05).
^{b}Missing cases were the result of (1) the detachment of section during immunostaining or (2) no cancer cells observed in the section (CDH17, 24 in stage I group end 27 in node-negative group; MUC13, 25 in node-negative group).
^{c}Missing cases resulted from no description of tumor size (n = 6).

The top 25 genes which were significantly up-regulated in IM or in SPEM are listed in Table E.

TABLE E
Top 25 genes significantly up-regulated in intestinal metaplasia (A) or in
spasmolytic polypeptide expressing metaplasia (B)

| | | | | | |
|---|---|---|---|---|---|
| | | | | Fold- | GO Cellular |
| No. | Symbol | Title | UniGene ID | up* | Component |

(A) intestinal metaplasia (IM)

| | | | | | |
|---|---|---|---|---|---|
| 1 | FABP1 | fatty acid binding protein 1, liver | Hs.380135 | 788.9 | cytoplasm |
| 2 | REG4 | regenerating islet-derived family, member 4 | Hs.660883 | 441.8 | extracellular |
| 3 | OLFM4 | olfactomedin 4 | Hs.508113 | 309.7 | extracellular |
| 4 | GDA | guanine deaminase | Hs.494163 | 264.2 | intracellular |
| 5 | DEFA5 | defensin, alpha 5, Paneth cell-specific | Hs.655233 | 260.3 | extracellular |
| 6 | ACE2 | angiotensin I converting enzyme (peptidyl- | Hs.178098 | 259.6 | extracellular |
| | | dipeptidase A) 2 | | | |
| 7 | DMBT1 | deleted in malignant brain tumors 1 | Hs.279611 | 253.2 | extracellular |
| 8 | PCK1 | phosphoenolpyruvate carboxykinase 1 | Hs.1872 | 215.6 | cytoplasm |
| 9 | CLCA1 | Chloride channel accessory 1 | Hs.194659 | 204.2 | integral to |
| | | | | | membrane |
| 10 | RBP2 | retinol binding protein 2, cellular | Hs.655516 | 193.9 | cytoplasm |
| 11 | KRT20 | keratin 20 | Hs.84905 | 190.6 | cytoplasm |
| | | | | | endoplasmic |
| 12 | HSD17B2 | hydroxysteroid (17-β) dehydrogenase 2 | Hs.162795 | 189.6 | reticulum |
| | | | | | membrane |
| 13 | MTTP | microsomal triglyceride transfer protein | Hs.195799 | 186.4 | soluble fraction |
| 14 | CDH17 | cadherin 17, LI cadherin (liver-intestine) | Hs.591853 | 156.3 | membrane |
| | | | | | fraction |
| 15 | SLC26A3 | solute carrier family 26, member 3 | Hs.1650 | 151.6 | membrane |
| | | | | | fraction |
| 16 | SI | sucrase-isomaltase (alpha-glucosidase) | Hs.429596 | 145.1 | Golgi apparatus |
| 17 | ANPEP | alanyl (membrane) aminopeptidase | Hs.1239 | 129.8 | soluble fraction |
| 18 | LGALS4 | lectin, galactoside-binding, soluble, 4 (galectin | Hs.5302 | 128.8 | cytosol |
| | | 4) | | | |
| 19 | SLC5A1 | solute carrier family 5 (sodium/glucose | Hs.1964 | 126.3 | integral to |
| | | cotransporter), member 1 | | | plasma |
| | | | | | membrane |
| 20 | MUC13 | mucin 13, cell surface associated | Hs.5940 | 115.1 | extracellular |
| 21 | SPINK4 | serine peptidase inhibitor, Kazal type 4 | Hs.555934 | 113.7 | extracellular |
| 22 | APOB | apolipoprotein B (including Ag(x) antigen) | Hs.120759 | 113.6 | extracellular |
| 23 | CPS1 | carbamoyl-phosphate synthetase 1, | Hs.149252 | 108.9 | mitochondrion |
| | | mitochondrial | | | |
| 24 | GBA3 | glucosidase, beta, acid 3 (cytosolic) | Hs.653107 | 103.8 | cytoplasm |
| 25 | PRSS7 | protease, serine, 7 (enterokinase) | Hs.149473 | 99.2 | brush border |

(B) spasmolytic polypeptide expressing metaplasia (SPEM)

| | | | | | |
|---|---|---|---|---|---|
| 1 | OLFM4 | olfactomedin 4 | Hs.508113 | 102.8 | extracellular |
| 2 | TFF1 | trefoil factor 1 | Hs.162807 | 31.2 | extracellular |
| 3 | GKN2 | gastrokine 2 | Hs.16757 | 26.4 | extracellular |
| 4 | TFF2 | trefoil factor 2 (spasmolytic protein 1) | Hs.2979 | 24.9 | extracellular |
| 5 | DPCR1 | diffuse panbronchiolitis critical region 1 | Hs.631993 | 23.3 | membrane |
| 6 | S100P | S100 calcium binding protein P | Hs.2962 | 22.6 | nucleus |
| 7 | FCGBP | Fc fragment of IgG binding protein | Hs.111732 | 21.6 | extracellular |
| 8 | LGALS4 | lectin, galactoside-binding, soluble, 4 (galectin | Hs.5302 | 17.6 | cytosol |
| | | 4) | | | |
| 9 | CEACAM5 | carcinoembryonic antigen-related cell adhesion | Hs.709196 | 16.8 | plasma |
| | | molecule 5 | | | membrane |
| 10 | GDA | guanine deaminase | Hs.494163 | 14.1 | intracellular |
| 11 | LYZ | lysozyme (renal amyloidosis) | Hs.524579 | 13.8 | extracellular |
| 12 | CFTR | cystic fibrosis transmembrane conductance | Hs.489786 | 13.7 | membrane |
| | | regulator | | | fraction |
| 13 | MUC5AC | mucin 5AC, oligomeric mucus/gel-forming | Hs.558950 | 13.3 | extracellular |
| 14 | KRT20 | keratin 20 | Hs.84905 | 12.0 | cytoplasm |
| 15 | ADH1C | alcohol dehydrogenase 1C (class I), gamma | Hs.654537 | 12.0 | cytoplasm |
| | | polypeptide | | | |
| 16 | AKR1B10 | aldo-keto reductase family 1, member B10 | Hs.116724 | 11.6 | cytoplasm |
| | | (aldose reductase) | | | |
| 17 | CDCA7 | cell division cycle associated 7 | Hs.470654 | 10.4 | nucleus |
| 18 | SLC5A1 | solute carrier family 5 (sodium/glucose | Hs.1964 | 10.2 | integral to plasma |
| | | cotransporter), member 1 | | | membrane |
| 19 | CYP2C18 | cytochrome P450, family 2, subfamily C, | Hs.511872 | 9.9 | endoplasmic |
| | | polypeptide 18 | | | reticulum |
| 20 | ELOVL6 | ELOVL family member 6, elongation of long | Hs.412939 | 9.7 | mitochondrion |
| | | chain fatty acids | | | |
| 21 | MUC13 | mucin 13, cell surface associated | Hs.5940 | 9.6 | extracellular |
| 22 | SLC6A14 | solute carrier family 6 (amino acid | Hs.522109 | 9.6 | integral to plasma |
| | | transporter), member 14 | | | membrane |
| 23 | AADAC | arylacetamide deacetylase (esterase) | Hs.506908 | 9.4 | endoplasmic |
| | | | | | reticulum |
| 24 | HSD17B2 | hydroxysteroid (17-beta) dehydrogenase 2 | Hs.162795 | 9.3 | endoplasmic |
| | | | | | reticulum |
| | | | | | membrane |
| 25 | GCNT3 | glucosaminyl (N-acetyl) transferase 3, mucin | Hs.194710 | 9.1 | Golgi membrane |
| | | type | | | |

*Fold change of genes in IM or SPEM, compared to chief cell

Identification of Markers for Gastric Metaplastic Lineages

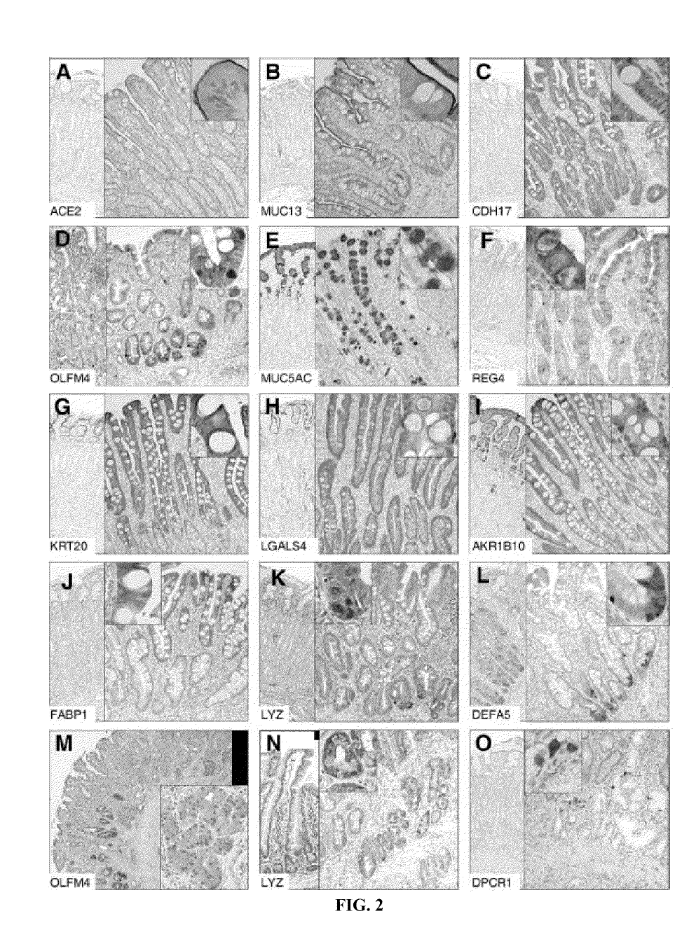

To examine the protein expression of selected genes in gastric metaplastic lineages, immunohistochemical staining was performed in IM, SPEM, and normal gastric fundic mucosa with more than 20 antibodies. Twelve proteins were expressed in different locations and distributions in IM, including (1) apical membranous expression in the luminal gland (ACE2) or in the entire gland (MUC13)²¹, (2) lateral membranous expression in the entire gland (CDH17)²² (3) scattered expression at the bases of gland (OLFM4)²³, (4) goblet staining in IM cells in the entire gland (MUC5AC, REG4)^{24,25}, (5) diffuse cytoplasmic expression in the luminal gland cells (KRT20)²⁶ or in the entire gland (LGALS4, AKR1B10, FABP1)²⁷, and (6) Paneth cell expression at the bases of glands (LYZ, DEFA5)²⁸. Three markers (ACE2, LGALS4, AKR1B10) had not been associated with IM previously. In addition, three proteins (OLFM4, LYZ, DPCR1) were found as novel SPEM markers. Out of 13 proteins described here, eight were completely negative in normal fundic mucosa, but five including MUC5AC, KRT20, LGALS4, AKR1B10 were expressed in normal foveolar cells. OLFM4 was expressed strongly in scattered cells at the bases of fundic glands and also showed variable diffuse staining in parietal cells (Table F, FIG. 2).

TABLE F
Expression profile of 13 proteins in normal fundus, intestinal metaplasia (IM),
spasmolytic polypeptide expressing metaplasia (SPEM), and gastric cancer

| | | Normal | IM | IM | | Gastric | | Intestinal | | Diffuse | |
|---|---|---|---|---|---|---|---|---|---|---|---|
| | Marker | fundus | pattern | location | SPEM | cancer | | type GC | | type GC | |
| 1 | MUC13 | — | Membranous | entire | − | 50% | (18/36) | 91%^{†} | (10/11) | 8% | (1/13) |
| | | | (apical) | | | | | | | | |
| 2 | CDH17 | FC+ | Membranous | entire | − | 41% | (17/42) | 42% | (11/26) | 42% | (5/12) |
| | | | (lateral) | | | | | | | | |
| 3 | OLFM4 | PC+ | scattered | basal | + | 41% | (17/42) | 46% | (12/26) | 25% | (3/12) |
| 4 | MUC5AC | FC+ | goblet | entire | − | 36% | (15/42) | 50% | (13/26) | 17% | (2/12) |
| 5 | KRT20 | FC+ | cytoplasmic | luminal | − | 36% | (13/36) | 73% | (8/11) | 0 | (0/13) |
| 6 | LGALS4* | FC+ | cytoplasmic | entire | − | 29% | (12/42) | 31% | (8/26) | 17% | (2/12) |
| 7 | AKR1B10* | — | cytoplasmic | entire | − | 29% | (12/42) | 35% | (9/26) | 17% | (2/12) |
| 8 | REG4 | — | goblet | entire | − | 17% | (7/42) | 19% | (5/26) | 8% | (1/12) |
| 9 | ACE2* | — | Membranous | luminal | − | 0 | (0/36) | 0 | (0/11) | 0 | (0/13) |
| | | | (apical) | | | | | | | | |
| 10 | FABP1 | — | cytoplasmic | entire | − | 0 | (0/42) | 0 | (0/26) | 0 | (0/12) |
| 11 | LYZ | — | Paneth cell | basal | + | 0 | (0/36) | 0 | (0/11) | 0 | (0/13) |
| 12 | DEFA5 | — | Paneth cell | basal | − | 0 | (0/36) | 0 | (0/11) | 0 | (0/13) |
| 13 | DPCR1 | — | — | — | + | 0 | (0/36) | 0 | (0/11) | 0 | (0/13) |

Abbreviations:
FC, foveolar cell;
PC, parietal cell;
IM, intestinal metaplasia;
SPEM, spasmolytic polypeptide expressing metaplasia;
GC, gastric cancer.
*novel markers for IM,
^{†}membranous pattern.
Statistically significant p-values (p < 0.05) are in boldface. sorted by the expression rate in gastric cancer tissue.

Expression Profile of Metaplastic Lineage Markers in Gastric Cancer

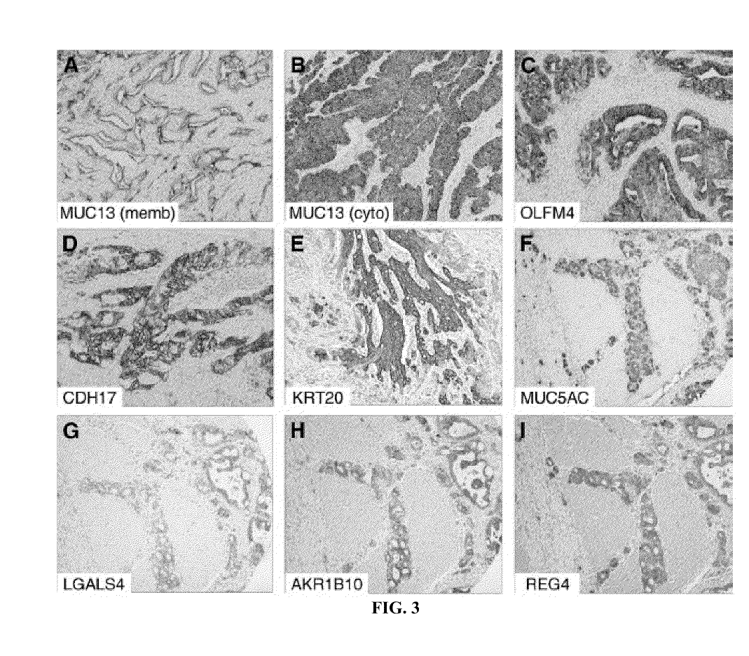

To identify the expression profiles in gastric cancer tissues of 13 proteins, which were expressed in IM or SPEM, immunohistochemical staining was performed in either the Vanderbilt-GC or SNUH-TA78 tissue arrays. MUC13 showed the highest expression rate in gastric cancers (50%), followed by OLFM4 (41%), CDH17 (41%), KRT20 (36%), MUC5AC (36%), LGALS4 (29%), AKR1B10 (29%), and REG4 (17%). ACE2, FABP1, DPCR1, LYZ, and DEFA5 were not expressed in any of the gastric cancers (FIG. 3). All of the proteins expressed in gastric cancers showed predominant expression in intestinal-type tumors, although the difference between intestinal-type and diffuse-type cancers did not reach statistical significance except for MUC13 and KRT20, both of which showed significantly higher expression in intestinal-type than in diffuse-type tumors (Table F).

Clinicopathologic and Prognostic Significance of MUC13, OLFM4, and CDH17 in Gastric Cancer Patients

For the proteins which were expressed in more than 40% of gastric cancers (MUC13, OLFM4, CDH17), the clinicopathologic and prognostic significance of the expression of these proteins were tested in the SNUH-2004-GC TMA (n=450; test set), and subsequently validated them in the SNUH-1996-GC TMA (n=502; validation set).

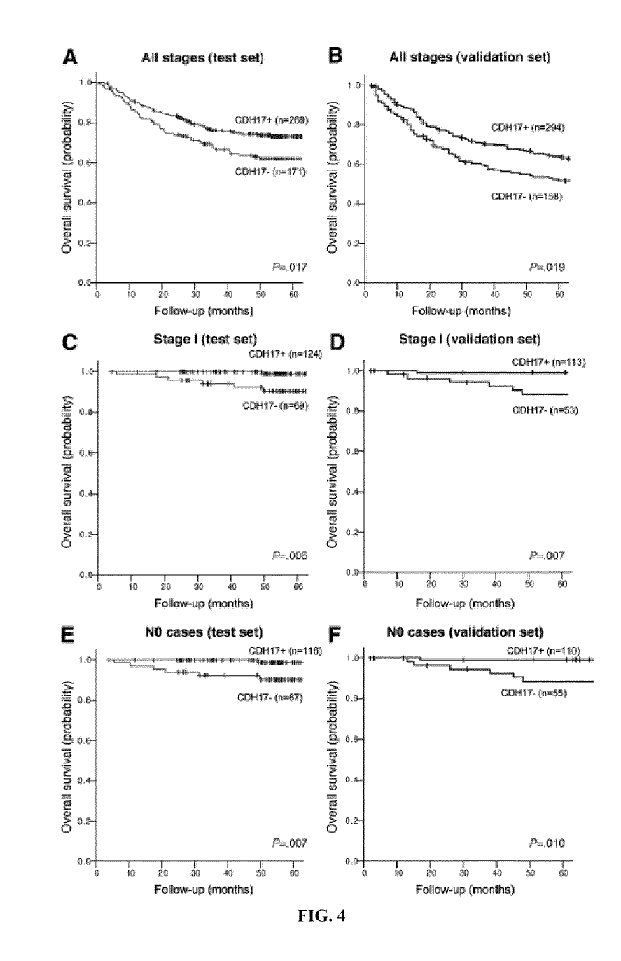

CDH17 was expressed in a membranous pattern in 61.1% and 65.0% of gastric cancers in the test and the validation set, respectively (FIG. 3D). CDH17 expression was significantly higher in intestinal-type cancers than in diffuse-type cancers. There was no significant difference in terms of lymphatic or venous invasion. The expression pattern according to TNM stage was not consistent between the test and the validation set (Table G). In the test set, the 5-year survival rate was significantly higher in patients with cancers expressing CDH17 (p=0.017, FIG. 4A). This survival difference was preserved only in patients with stage I disease (p=0.006, FIG. 4C), not in stage II or more (data not shown). Similarly, this survival difference was preserved only in patients with node-negative disease (p=0.007, FIG. 4E), not in cases with node-positive disease (data not shown). These prognostic impacts of CDH17 were reproduced in the validation set (FIGS. 4B, D, F).

TABLE G
Expression profile of CDH17, MUC13, and OLFM4 in gastric cancer
according to the clinicopathologic characteristics*
1) CDH17 (membranous expression)

| | | 2004 (n = 440) | p-value | 1996 (n = 452) | p-value |
|---|---|---|---|---|---|
| Total | | 61.1% (269/440) | | 65.0% (294/452) | |
| Lauren | Intestinal | 68.2% (122/179) | 0.037 | 73.5% (150/204) | 0.002 |
| | Diffuse | 54.1% (98/181) | | 59.1% (137/232) | |
| | Mixed | 61.3% (49/80) | | 40% (6/15) | |
| TNM | I | 64.2% (124/193) | 0.025 | 68.1% (113/166) | 0.28 |
| | II | 69.4% (59/85) | | 68.6% (70/102) | |
| | III | 48.1% (39/81) | | 62.8% (71/113) | |
| | IV | 58.0% (47/81) | | 56.3% (40/71) | |

| | | | |
|---|---|---|---|
| 2004 (n = 433) | p-value | 1996 (n = 472) | p-value |

2) MUC13 (membranous expression)

| | | | | | |
|---|---|---|---|---|---|
| Total | | 44.1% (191/433) | | 44.5% (210/472) | |
| Lauren | Intestinal | 79.0% (139/176) | <0.001 | 80.0% (160/200) | <0.001 |
| | Diffuse | 8.4% (15/179) | | 16.9% (43/254) | |
| | Mixed | 47.4% (37/78) | | 37.5% (6/16) | |
| TNM | I | 53.7% (101/188) | 0.003 | 51.8% (88/170) | 0.044 |
| | II | 41.7% (35/84) | | 45.4% (49/108) | |
| | III | 35.8% (29/81) | | 40.5% (49/121) | |
| | IV | 32.5% (26/80 | | 33.3% (24/72) | |

3) MUC13 (cytoplasmic expression)

| | | | | | |
|---|---|---|---|---|---|
| Total | | 30.7% (133/433) | | 25.4% (120/472) | |
| Lauren | Intestinal | 18.2% (32/176) | <0.001 | 23.5% (47/200) | 0.431 |
| | Diffuse | 40.8% (73/179) | | 27.6% (70/254) | |
| | Mixed | 35.9% (28/78) | | 12.5% (2/16) | |
| TNM | I | 25.0% (47/188) | 0.033 | 22.4% (38/170) | 0.005 |
| | II | 27.4% (23/84) | | 19.4% (21/108) | |
| | III | 38.3% (31/81) | | 24.8% (30/121) | |
| | IV | 40.0% (32/80) | | 41.7% (30/72) | |

4) OLFM4 (cytoplasmic expression)

| | | 2004 (n = 435) | p-value | 1996 (n = 476) | p-value |
|---|---|---|---|---|---|
| Total | | 26.0% (113/435) | | 27.1% (129/476) | |
| Lauren | Intestinal | 32.4% (57/176) | 0.030 | 32.7% (67/205) | 0.11 |
| | Diffuse | 19.4% (35/180) | | 23.3% (59/253) | |
| | Mixed | 26.6% (21/79) | | 18.8% (3/16) | |
| TNM | I | 30.7% (58/189) | 0.17 | 28.1% (48/171) | 0.98 |
| | II | 22.6% (19/84) | | 25.7% (28/109) | |
| | III | 18.5% (15/81) | | 27.5% (33/120) | |
| | IV | 25.9% (21/81) | | 26.7% (20/75) | |

*Missing cases were resulted from (1) the detachment of section during immunostaining or (2) no cancer cells observed in the section.
Statistically significant p-values (p < 0.05) are in boldface.

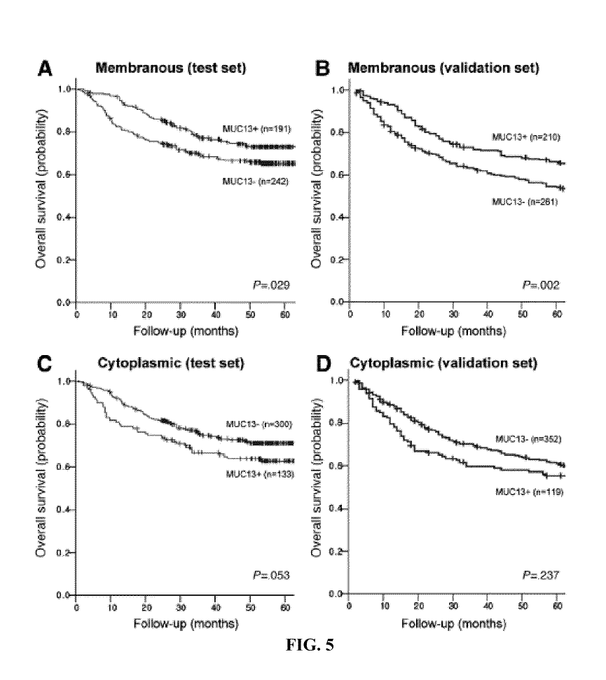

Two different expression patterns were observed for MUC13: membranous and diffuse cytoplasmic. The membranous pattern of MUC13 staining was observed in 44.1% of gastric cancers in the test set and in 44.5% of the validation set, respectively (FIG. 3A), and its expression was significantly higher in intestinal-type tumors and in earlier TNM stage in both sets. The diffuse cytoplasmic pattern of MUC13 was expressed in 30.7% of the test set cases and in 25.4% of the validation set (FIG. 3B). In contrast with the membranous pattern, the cytoplasmic expression was significantly higher in advanced TNM stages (Table G). Five-year disease-specific survival rate was significantly higher in cases expressing membranous pattern of MUC13 in the test set (p=0.029, FIG. 5A) and in the validation set (p<0.001, FIG. 5B). In contrast, the prognostic impact of cytoplasmic expression of MUC13 showed a tendency towards decreased survival, although it did not reach statistical significance in either set (FIGS. 5C and 4D). These findings support the concept that redistribution of MUC13 off the membrane is related to poorer patient outcome.

OLFM4 was expressed in a diffuse cytoplasmic pattern in 26.0% of the test set cases and in 27.1% of the validation set (FIG. 3C). OLFM4 expression showed a tendency towards higher expression in intestinal-type cancers than in diffuse-type cancers. There was no significant difference in relation to TNM stage or lymphatic or venous invasion (Table G). The prognostic impact of OLFM4 expression was not observed in the entire gastric cancer patient cohort. Although 5-year disease-specific survival rate was significantly lower in OLFM4 positive cases in stage I disease in the test set (p=0.018), this significance was not observed in the validation set (p=0.889).

Multivariate Analysis and Subgroup Analysis

When a multivariate analysis of all patients was performed, only TNM classifications were revealed as an independent prognostic factor for survival (data not shown). However, in the case of patients with stage I disease, as with TNM stage and venous invasion, the expression of CDH17 was also revealed as an independent prognostic factor for disease-specific survival (Table H). In addition, in the case of patients with node-negative, curatively resected cancers, as with T-classification, the expression of CDH17 was also an independent prognostic factor for disease-specific survival (Table H). These independent prognostic impacts of CDH17 in stage I or in node-negative gastric cancer patients were observed both in the test set and in the validation set, even when analyzed separately (data not shown).

TABLE H
Cox Multivariate Analysis for Disease-Specific Survival in
Subgroups of Gastric Cancer Patients.

| | | | | |
|---|---|---|---|---|
| | | No. of | | |
| Factor | Variable | patients | 95% CI | P value |

Curatively resected, stage I gastric cancer (n = 383)

| | | | | |
|---|---|---|---|---|
| TNM stage | la/lb | 208/175 | 2.348-135.597 | .005^{a} |
| Venous invasion | No/yes | 371/12 | 2.097-69.908 | .005^{a} |
| CDH17^{b} | No/yes | 122/237 | 2.858-38.970 | <.001^{a} |

Curatively resected, node-negative gastric cancer (n = 378)

| | | | | |
|---|---|---|---|---|
| T-stage | T1 | 208 | — | .002^{a} |
| | T2 | 150 | 2.178-133.110 | .007 |
| | T3 | 18 | 0.562-164.628 | .118 |
| | T4 | 2 | 18.452-9976.457 | <.001 |
| CDH17^{b} | No/yes | 123/228 | 1.521-16.108 | .008^{a} |
| MUC13 (memb)^{b} | No/yes | 180/173 | 0.545-5.932 | .335 |
| Size (cm)^{c} | <5/≧5 | 269/103 | 0.282-2.643 | .797 |

^{a}Statistically significant P values (P < .05).
^{b}Missing cases were the result of (1) the detachment of section during immunostaining or (2) no cancer cells observed in the section (CDH17, 24 in stage I group and 27 in node-negative group; MUC13, 25 in node-negative group).
^{c}Missing cases resulted from no description of tumor size (n = 6).

In a subgroup analysis according to the tumor location, the expression of CDH17 and the membranous expression of MUC13 showed a better prognosis only in antral cancers (p=0.006 and p=0.002, respectively), but not in the non-antral cancers. According to Lauren classification, the expression of CDH17 showed a better prognosis only in diffuse type (p=0.014), but not in intestinal type cancers. In contrast, cytoplasmic expression of MUC13 showed worse survival only in intestinal type cancers (p=0.018), not in diffuse type tumors.

Discussion

Perioperative or postoperative chemotherapy is generally recommended for the treatment of advanced gastric cancer.^{3,29} However, for stage I gastric cancer, which has a 20˜30% 5-year recurrence rate, appropriate criteria for adjuvant chemotherapy have not been available. In contrast, in the early-stage, node-negative breast cancer, a number of prognostic markers are used in the clinical setting to select candidates for adjuvant treatment.³⁰ The results in the present investigation suggest that CDH17, an independent prognostic marker for stage I or node-negative gastric cancer, is a useful biomarker for selection of adjuvant chemotherapy in early-stage gastric cancer patients, although further large-scale prospective studies are required.

To identify genes associated with the early neoplastic processes, the present investigation was focused upon on the identification of biomarkers for metaplastic lineages. IM is established as a possible premalignant lineage for gastric cancer, although many questions remain regarding its direct involvement in cancer pathogenesis.^{10,31} In contrast, the role of SPEM as a preneoplastic process has received attention only recently. Animal studies have suggested that SPEM originates from transdifferentiation of chief cells in fundic glands, and can evolve into dysplasia in the presence of a chronic inflammatory process.^{11,12} Furthermore, investigations in Mongolian gerbils infected with H. pylori and in amphiregulin knock-out mice have supported the concept that SPEM evolves first following loss of parietal cells, while IM develops from SPEM as a secondary metaplasia.^{17,18} This relationship between SPEM and IM is supported by the present cDNA microarray data of IM and SPEM, where 45 (94%) of 48 probes significantly up-regulated in SPEM were also significantly up-regulated in IM. Indeed, for a number of these genes, a progression of increased expression from chief cells to SPEM and from SPEM to IM was observed. Nevertheless, caution is merited in the interpretation of transcript expression profiles based on microarray. Thus, prominent elevations in the expression of TFF1 and GKN2 transcripts in SPEM and IM were also noted. However, while protein immunostaining for TFF1 was observed in normal surface cells, in the same sections no staining of either SPEM or IM (data not shown) was seen. It is therefore useful to validate that elevations in mRNA expression are reflected in changes in protein expression.

Among the genes identified in metaplasia, an independent prognostic biomarker, CDH17, was successfully documented, especially in early-stage gastric cancer. CDH17 (cadherin-17; liver-intestine cadherin) is a structurally unique member of the cadherin superfamily, and acts as a functional Ca²⁺-dependent homophilic cell adhesion molecule.³² In humans, CDH17 is expressed exclusively on the basolateral surface of hepatocytes and enterocytes, as confirmed in the present study (FIG. 2D). After the first report of CDH17 as an IM marker by Grotzinger et al,²² several investigations have evaluated CDH17 expression in gastric cancer. CDH17 was expressed in 60-78% of gastric cancer tissues with intestinal-type predominance, similar to the data here.^{33,34} However, the relationship between CDH17 expression and cancer stage or patients' survival was inconclusive. Park and colleagues evaluated the CDH17 expression in more than 200 gastric cancer tissue samples, and reported that it was highly expressed in earlier TNM stages.³⁵ However, others reported that its expression was much higher in advanced cancer stages.^{33,36} As a prognostic factor, the previously available data were limited, but CDH17 expression showed a tendency towards an unfavorable indicator for survival.^{33,36} In the study, the mRNA expression of CDH17 was increased 156.3-fold in IM and 7.8-fold in SPEM, compared to normal chief cells. Also, CDH17 was expressed in 61-65% of human gastric cancers with no correlation with TNM stage. The favorable impact of CDH17 expression on a prognosis of stage I or node-negative gastric cancer patients, shown in both the test set and the validation set in the study, may reflect the role of this protein in the maintenance of polarity and normal cell-to-cell adhesion.

MUC13 (mucin 13) was also revealed as a novel prognostic marker. MUC13 gene encodes a transmembrane mucin that is specifically expressed in digestive tract tissues.³⁷ Over-expression of MUC13 was reported previously in several cancers including gastric, colorectal, and ovarian cancers.^{21,38,39} The results showed that two distinct staining patterns (membranous and cytoplasmic) exist for MUC13 in gastric cancer tissues, a finding similar to the previous report on colorectal cancer tissues.³⁸ The membranous pattern was expressed in gastric cancer with intestinal-type histology, an early stage, and a favorable outcome, while the cytoplasmic pattern correlated with advanced stage. A significant reverse correlation was observed in membranous and cytoplasmic expression patterns of MUC13 in the study (R²=−0.173, p<0.001 in Pearson's correlation). The underlying mechanism of this distinct staining pattern for MUC13 in gastric cancers will require further investigation.

OLFM4 (olfactomedin 4; hGC-1, GW112) is a member of a growing olfactomedin protein family.⁴⁰ Some studies indicate that OLFM4 may act as an anti-apoptotic protein that promotes tumor growth.⁴¹ OLFM4 is normally expressed in small intestine, colon, and prostate, and its mRNA was over-expressed in gastric and colorectal cancers.^{4,44} Recently, OLFM4 was identified as a stem cell marker in the human intestine where it is co-expressed with Lgr5 which was reported as stem cell marker in the pyloric glands, not in the fundic glands.^{42,43} Liu et al. first reported the expression of OLFM4 in IM and in 65% of intestinal-type gastric cancer.²³ Recently, Oue et al reported the serum ELISA data of OLFM4 in gastric cancer patients as well as its prognostic impact on survival.⁴⁵ In those studies, OLFM4 was revealed as a favorable prognostic marker in intestinal-type gastric cancer, in contrast with the present study. The data indicated that OLFM4 mRNA expression was increased 309.7-fold in IM and 102.8-fold in SPEM. Also, OLFM4 immunostaining was detected in 32% of intestinal-type gastric cancers and its prognostic impact was not consistent between the test set and the validation set. Subgroup analysis according to Lauren classification also did not show any prognostic impact of OLFM4 in the study. More studies are needed to validate the clinical implications of OLFM4 in gastric cancer.

In summary, a number of putative biomarkers were identified for the metaplastic process in the stomach. CDH17 is an independent prognostic factor in patients with stage I or node-negative gastric cancer.

Throughout this document, various references are mentioned. All such references are incorporated herein by reference, including the references set forth in the following list:

### REFERENCES

1. Parkin D M, Bray F, Ferlay J, Pisani P. Global cancer statistics, 2002. CA Cancer J Clin 55:74-108, 2005
2. Sasako M, Sano T, Yamamoto S, et al. D2 lymphadenectomy alone or with para-aortic nodal dissection for gastric cancer. N Engl J Med 359:453-462, 2008.
3. Sakuramoto S, Sasako M, Yamaguchi T, et al. Adjuvant chemotherapy for gastric cancer with S-1, an oral fluoropyrimidine. N Engl J Med 357:1810-1820, 2007.
4. Yasui W, Oue N, Aung P P, et al. Molecular-pathological prognostic factors of gastric cancer: a review. Gastric Cancer 8:86-94, 2005.
5. Hasegawa S, Furukawa Y, Li M, et al. Genome-wide analysis of gene expression in intestinal-type gastric cancers using a complementary DNA microarray representing 23,040 genes. Cancer Res 62:7012-7017, 2002.
6. Leung S Y, Chen X, Chu KM, et al. Phospholipase A2 group IIA expression in gastric adenocarcinoma is associated with prolonged survival and less frequent metastasis. Proc Natl Acad Sci USA 99:16203-16208, 2002.
7. Boussioutas A, Li H, Liu J, et al. Distinctive patterns of gene expression in premalignant gastric mucosa and gastric cancer. Cancer Res 63:2569-2577, 2003.
8. Oue N, Hamai Y, Mitani Y, et al. Gene expression profile of gastric carcinoma: identification of genes and tags potentially involved in invasion, metastasis, and carcinogenesis by serial analysis of gene expression. Cancer Res 64:2397-2405, 2004.
9. Vecchi M, Nuciforo P, Romagnoli S, et al. Gene expression analysis of early and advanced gastric cancers. Oncogene 26:4284-4294, 2007.
10. Gutiérrez-González L, Wright NA. Biology of intestinal metaplasia in 2008: more than a simple phenotypic alteration. Dig Liver Dis 40:510-522, 2008.
11. Wang T C, Goldenring J R, Dangler C, et al. Mice lacking secretory phospholipase A2 show altered apoptosis and differentiation with Helicobacter felis infection. Gastroenterology 114:675-89, 1998.
12. Houghton J, Stoicov C, Nomura S, et al. Gastric cancer originating from bone marrow-derived cells. Science 306:1568-1571, 2004.
13. Schmidt P H, Lee J R, Joshi V, et al. Identification of a metaplastic cell lineage associated with human gastric adenocarcinoma. Lab Invest 79:639-646, 1999.
14. Yamaguchi H, Goldenring J R, Kaminishi M, et al. Identification of spasmolytic polypeptide expressing metaplasia (SPEM) in remnant gastric cancer and surveillance postgastrectomy biopsies. Dig Dis Sci 47:573-578, 2002.
15. Nam K T, Varro A, Coffey R J, et al. Potentiation of oxyntic atrophy-induced gastric metaplasia in amphiregulin-deficient mice. Gastroenterology 132:1804-1819, 2007.
16. Nozaki K, Ogawa M, Williams J A, et al. A molecular signature of gastric metaplasia arising in response to acute parietal cell loss. Gastroenterology 134:511-522, 2008.
17. Nam K T, Lee H J, Mok H, et al. Amphiregulin-deficient mice develop spasmolytic polypeptide expressing metaplasia and intestinal metaplasia. Gastroenterology 136: 1288-1296, 2009.
18. Yoshizawa N, Takenaka Y, Yamaguchi H, et al. Emergence of spasmolytic polypeptide-expressing metaplasia in Mongolian gerbils infected with Helicobacter pylori. Lab Invest 87:1265-1276, 2007.
19. Hochberg Y, Benjamini Y. More powerful procedures for multiple significance testing. Stat Med 9:811-818, 1990.
20. Leys C M, Nomura S, Rudzinski E, et al. Expression of Pdx-1 in human gastric metaplasia and gastric adenocarcinoma. Hum Pathol 37:1162-1168, 2006.
21. Shimamura T, Ito H, Shibahara J, Watanabe A, et al. Overexpression of MUC13 is associated with intestinal-type gastric cancer. Cancer Sci 96:265-273, 2005.
22. Grötzinger C, Kneifel J, Patschan D, et al. LI-cadherin: a marker of gastric metaplasia and neoplasia. Gut 49:73-81, 2001.
23. Liu W, Zhu J, Cao L, et al. Expression of hGC-1 is correlated with differentiation of gastric carcinoma. Histopathology 51:157-165, 2007.
24. Reis C A, David L, Correa P, et al. Intestinal metaplasia of human stomach displays distinct patterns of mucin (MUC1, MUC2, MUC5AC, and MUC6) expression. Cancer Res 59:1003-1007, 1999.
25. Oue N, Mitani Y, Aung PP, et al. Expression and localization of Reg IV in human neoplastic and non-neoplastic tissues: Reg IV expression is associated with intestinal and neuroendocrine differentiation in gastric adenocarcinoma. J Pathol 207:185-198, 2005.
26. Ormsby A H, Vaezi M F, Richter J E et al. Cytokeratin immunoreactivity patterns in the diagnosis of short-segment Barrett's esophagus. Gastroenterology 119:683-690, 2000.
27. Hashimoto T, Kusakabe T, Watanabe K, et al. Liver-type fatty acid-binding protein is highly expressed in intestinal metaplasia and in a subset of carcinomas of the stomach without association with the fatty acid synthase status in the carcinoma. Pathobiology 71:115-122, 2004.
28. Ouellette A J. Paneth cells and innate immunity in the crypt microenvironment. Gastroenterology 113:1779-1784, 1997.
29. Cunningham D, Allum W H, Stenning S P, et al. Perioperative chemotherapy versus surgery alone for resectable gastroesophageal cancer. N Engl J Med 355:11-20, 2006.
30. Mirza A N, Mirza N Q, Vlastos G, et al. Prognostic factors in node-negative breast cancer: a review of studies with sample size more than 200 and follow-up more than 5 years. Ann Surg 235:10-26, 2002.
31. Meining A, Morgner A, Miehlke S, et al. Atrophy-metaplasia-dysplasia-carcinoma sequence in the stomach: a reality or merely a hypothesis? Best Pract Res Clin Gastroenterol 15:983-998, 2001.
32. Gessner R, Tauber R. Intestinal cell adhesion molecules. Liver-intestine cadherin. Ann N Y Acad Sci 915:136-143, 2000.
33. Ito R, Oue N, Yoshida K, Kunimitsu K, et al. Clinicopathological significant and prognostic influence of cadherin-17 expression in gastric cancer. Virchows Arch 447:717-722, 2005.
34. Ko S, Chu K M, Luk J M, et al. CDX2 co-localizes with liver-intestine cadherin in intestinal metaplasia and adenocarcinoma of the stomach. J Pathol 205:615-622, 2005.
35. Park S S, Kang S H, Park J M, et al. Expression of liver-intestine cadherin and its correlation with lymph node metastasis in gastric cancer: can it predict N stage preoperatively? Ann Surg Oncol 14:94-99, 2007.
36. Ge J, Chen Z, Wu S, et al. A clinicopathological study on the expression of cadherin-17 and caudal-related homeobox transcription factor (CDX2) in human gastric carcinoma. Clin Oncol (R Coll Radiol) 20:275-283, 2008.
37. Williams S J, Wreschner D H, Tran M, et al. Muc13, a novel human cell surface mucin expressed by epithelial and hemopoietic cells. J Biol Chem 276:18327-1836, 2001.
38. Walsh M D, Young J P, Leggett B A, et al. The MUC13 cell surface mucin is highly expressed by human colorectal carcinomas. Hum Pathol 38:883-892, 2007.
39. Chauhan S C, Vannatta K, Ebeling M C, et al. Expression and functions of transmembrane mucin MUC13 in ovarian cancer. Cancer Res 69:765-774, 2009.
40. Zhang J, Liu W L, Tang D C, et al. Identification and characterization of a novel member of olfactomedin-related protein family, hGC-1, expressed during myeloid lineage development. Gene 283:83-93, 2002.
41. Zhang X, Huang Q, Yang Z, et al. GW112, a novel antiapoptotic protein that promotes tumor growth. Cancer Res 64:2474-2481, 2004.
42. van der Flier L G, Haegebarth A, Stange D E, et al. OLFM4 is a robust marker for stem cells in human intestine and marks a subset of colorectal cancer cells. Gastroenterology 137:15-17, 2009.
43. Barker N, Huch M, Kujala P, et al. Lgr5^{+ve} stem cells drive self-renewal in the stomach and build long-lived gastric units in vitro. Cell Stem Cell 6:25-36, 2010.
44. Koshida S, Kobayashi D, Moriai R, et al. Specific overexpression of OLFM4 (GW112/HGC-1) mRNA in colon, breast and lung cancer tissues detected using quantitative analysis. Cancer Sci 98:315-320, 2007.
45. Oue N, Sentani K, Noguchi T, et al. Serum olfactomedin 4 (GW112, hGC-1) in combination with Reg IV is a highly sensitive biomarker for gastric cancer patients. Int J Cancer 125:2383-2392, 2009.
46. Fukumoto S, Yamauchi N, Moriguchi H, et al. Overexpression of the aldo-keto reductase family protein AKR1B10 is highly correlated with smokers' non-small cell lung carcinomas. Clin Cancer Res 11:1776-1785, 2005.

What is claimed is:

1. A method for diagnosing and/or monitoring a gastric cancer in a subject, comprising:
(a) providing a biological sample from the subject;
(b) determining an amount in the sample of at least one biomarker, selected from the group consisting of: CDH17 and OLFM4; and
(c) comparing the amount of the at least one biomarker in the sample, if present, to a control level of the at least one biomarker.

2. The method of claim 1, further comprising determining an amount in the sample of a MUC13 biomarker.

3. The method of claim 1, further comprising determining an amount in the sample of at least one biomarker, selected from the group consisting of: FABP1, REG4, GDA, DEFA5, ACE2, DMBT1, PCK1, CLCA1, RBP2, KRT20, HSD17B2, MTTP, SLC26A3, SI, ANPEP, LGALS4, SLC5A1, MUC13, SPINK4, APOB, CPS1, GBA3, PRSS7, TFF1, GKN2, TFF2, DPCR1, S100P, FCGBP, LGALS4, CEACAM5, GDA, LYZ, CFTR, MUC5AC, KRT20, ADH1C, AKR1B10, CDCA7, SLC5A1, CYP2C18, ELOVL6, MUC13, SLC6A14, AADAC, HSD17B2, and GCNT3.

4. The method of claim 3, wherein the subject is diagnosed as having the gastric cancer or a risk thereof if there is a measurable difference in the amount of the at least one biomarker in the sample as compared to the control level.

5. The method of claim 3, further comprising, providing a series of biological samples over a time period from the subject; and determining any measurable change in the amount of the at least one biomarker in each of the biological samples to thereby determine whether to initiate or continue prophylaxis or therapy of the cancer.

6. The method of claim 5, wherein the series of biological samples comprises a first biological sample collected prior to initiation of the prophylaxis or treatment for the gastric cancer and a second biological sample collected after initiation of the prophylaxis or treatment.

7. The method of claim 1, wherein the subject is diagnosed as having the gastric cancer or a risk thereof if there is a measurable difference in the amount of the at least one biomarker in the sample as compared to the control level.

8. The method of claim 7, wherein the gastric cancer is a precancerous or cancerous pathology selected from the group consisting of intestinal metaplasia (IM), spasmolytic-polypeptide expressing metaplasia (SPEM), a stage I gastric cancer, a stage-II gastric cancer, a stage-III gastric cancer, a stage-IV gastric cancer, a gastric adenocarcinoma, and a node-negative gastric cancer.

9. The method of claim 1, further comprising, providing a series of biological samples over a time period from the subject; and determining any measurable change in the amount of the at least one biomarker in each of the biological samples to thereby determine whether to initiate or continue prophylaxis or therapy of the cancer.

10. The method of claim 9, wherein the gastric cancer is a precancerous or cancerous pathology selected from the group consisting of intestinal metaplasia (IM), spasmolytic-polypeptide expressing metaplasia (SPEM), a stage I gastric cancer, a stage-II gastric cancer, a stage-III gastric cancer, a stage-IV gastric cancer, a gastric adenocarcinoma, and a node-negative gastric cancer.

11. The method of claim 9, wherein the series of biological samples comprises a first biological sample collected prior to initiation of the prophylaxis or treatment for the gastric cancer and a second biological sample collected after initiation of the prophylaxis or treatment.

12. The method of claim 1, wherein the gastric cancer is a precancerous or cancerous pathology selected from the group consisting of intestinal metaplasia (IM), spasmolytic-polypeptide expressing metaplasia (SPEM), a stage I gastric cancer, a stage-II gastric cancer, a stage-III gastric cancer, a stage-IV gastric cancer, a gastric adenocarcinoma, and a node-negative gastric cancer.

13. The method of claim 1, wherein the biological sample comprises blood, serum, plasma, gastric secretions, a gastrointestinal biopsy sample, a sample obtained at the time or gastrointestinal resection, microdissected cells from a gastrointestinal biopsy of resection, gastrointestinal cells sloughed into the gastrointestinal lumen, and gastrointestinal cells recovered from stool.

14. The method of claim 1, wherein determining the amount of the at least one biomarker comprises one or more techniques selected from:
(a) determining an amount of mRNA of the at least one biomarker in the biological sample using an RNA measuring assay; and
(b) determining an amount of a polypeptide of the at least one biomarker in the biological sample using a protein measuring assay.

15. The method of claim 14, wherein the RNA measuring assay comprises an array of RNA hybridization probes or a quantitative polymerase chain reaction assay.

16. The method of claim 14, wherein the protein measuring assay comprises mass spectrometry (MS) analysis, immunoassay analysis, or both.

17. The method of claim 16, wherein the immunoassay analysis comprises one or more antibodies that selectively bind the at least one biomarker.

18. The method of claim 1, wherein determining the amount of the at least one biomarker comprises immunohistochemical staining of the at least one biomarker in the biological sample.

19. The method of claim 18, wherein the biological sample is selected from a gastrointestinal biopsy sample, a sample obtained at the time of gastrointestinal resection, and microdissected cells from a gastrointestinal biopsy or resection,

20. The method of claim 1, further comprising selecting a treatment or modifying a treatment for the cancer based on the amount of the at least one biomarker determined.

21. A kit for diagnosing or monitoring a gastric cancer in a subject, the kit comprising a probe for selectively binding each of at least one biomarker selected from the group consisting of: CDH17 and OLFM4.

22. The kit of claim 21, further comprising a probe for selectively binding each of at least one biomarker selected from the group consisting of: FABP1, REG4, GDA, DEFA5, ACE2, DMBT1, PCK1, CLCA1, RBP2, KRT20, HSD17B2, MTTP, SLC26A3, SI, ANPEP, LGALS4, SLC5A1, MUC13, SPINK4, APOB, CPS1, GBA3, PRSS7, TFF1, GKN2, TFF2, DPCR1, S100P, FCGBP, LGALS4, CEACAM5, GDA, LYZ, CFTR, MUC5AC, KRT20, ADH1C, AKR1B10, CDCA7, SLC5A1, CYP2C18, ELOVL6, MUC13, SLC6A14, AADAC, HSD17B2, and GCNT3

23. The kit of claim 21, wherein the probes are bound to a substrate.

24. The kit of claim 21, wherein the probes are labeled to allow for detecting the binding of the probes to the at least one biomarker.

25. The kit of claim 21, wherein the probes are RNA hybridization probes.

26. The kit of claim 21, wherein the probes are antibodies.
